(19) United States
(12) Patent Application Publication
(10) Pub. No.: US 2014/0079740 A1
(43) Pub. Date: Mar. 20, 2014

(54) ORAL TRANSMUCOSAL ADMINSTRATION FORMS OF S-KETAMINE

(71) Applicant: ClinPharm Support GmbH, Amriswil (CH)

(72) Inventor: Zoser B. Salama, Ravensburg (DE)

(73) Assignee: ClinPharm Support GmbH, Amriswil (CH)

(21) Appl. No.: 13/957,974

(22) Filed: Aug. 2, 2013

(30) Foreign Application Priority Data
Aug. 2, 2012 (EP) 12075089.8

(51) Int. Cl. A61K 9/00; A61K 45/06; A61K 31/135

(52) U.S. Cl. CPC A61K 9/006; A61K 31/135; A61K 45/06
USPC 424/400; 514/647

(57) ABSTRACT

The present invention relates to methods and compositions for the treatment of pain, in a preferred embodiment relating to the oral transmucosal administration of S-Ketamine, its salts or derivatives.

The present invention relates to methods and compositions for the treatment of pain. The treatment of pain encompasses more specifically the prophylaxis, prevention, reduction, attenuation, elimination and/or therapy the symptoms of said acute, chronic break-through cancer pain (BTCP), complex regional pain syndrome (CRPS), refractory cancer pain, neutopathic pain, post traumatic syndrome (PTSD) and/or ischaematic limb pain. More particularly the invention relates to transmucosal, transbucal, sublingual, fast dissolving oral films, fast integrating tablets, flat film forming dosage form administration of S-(+) ketamine, its salts and/or derivatives.

The present invention is based on the surprising and unexpected development that transmucosal, transbucal, sublingual, fast dissolving oral films, fast integrating tablets, flat film forming dosage form administration of S-(+) ketamine, its salts and/or derivatives, can prophylaxis, prevent, attenuate, reduce, eliminate and/or therapeutical treat the symptoms of said acute, chronic break-through cancer pain (BTCP), complex regional pain syndrome (CRPS), refractory cancer pain, neuropathic pain, post traumatic syndrome (PTSD) and/or ischaematic limb pain.

### BACKGROUND OF THE INVENTION

Pain is an unpleasant sensation localized to a part of the body. It is often described in terms of a penetrating or tissue-destructive process (e.g., stabbing, burning, twisting, tearing, squeezing) and/or a bodily or emotional reaction (e.g., terrifying, nauseating, sickening). Management of patients suffering pain is intellectually and emotionally challenging, whether the pain be acute or chronic.

Breakthrough pain is one of the most common and feared symptoms of cancer. Many patients suffering from cancer have more than one type of pain, not all pains are, however, due to cancer itself. At least two-third of the patients suffering from advanced cancer report pain (WHO 1996). Pain relief is achieved adequately in a majority of cancer patients using the WHO guidelines. Pain in cancer patients has two components. One is persistent pain that lasts for more than 12 hours/day. However, in addition to persistent pain, patients may also experience transient exacerbations of significant and severe pain on a background of otherwise well controlled pain. These severe flare ups of pain are called breakthrough pain as the pain breaks through the regular pain medication. Specific characteristics that further define breakthrough pain include it's relation to the fixed dose of opioid medication, temporal features, precipitating events and its predictability. This breakthrough pain has an incidence of about 40-86% as reported in various studies.

### Consequences of Breakthrough Pain

Untreated breakthrough pain has significant consequences for individual patients, their caregivers and the healthcare system. Without treatment, flares of breakthrough pain can harm a person's sense of well being, interfere with daily activities, interrupt disease related treatment schedules and make it even more difficult to treat persistent pain. As fear of breakthrough pain events grows, patients tend to remain sedentary thus exacerbating physical deconditioning and pain related disability. Effective treatment of breakthrough pain is not only good practice but also cost effective as past studies have shown that effective breakthrough pain relief decreases cost of overall treatment by five times. Therefore, although assessment of breakthrough pain and its treatment may initially increase the cost of treatment, overall it will be less costly.

### Pharmacological Approaches for the Treatment of Breakthrough Pain

Anti-inflammatory drugs: Additive analgesia produced by NSAIDS and steroidal anti-inflammatory drugs is useful in painful bone metastases, mucosal and skin lesions. While long acting NSAIDS allowing once or twice daily dosing is preferred in patients taking multiple drugs, rescue doses of particular formulations of NSAIDS (sublingually or parentally) is preferred in treating breakthrough pain particularly when side effects from rescue doses of opioids become intolerable.

Opioids: Patients with breakthrough pain are usually treated with an opioid drug. The use of as needed analgesia with rescue doses of opioids to treat established breakthrough pain or prevent anticipated episode is the current gold standard of management in spite of the fact that the pharmacokinetics of oral opioid does not match the requirements of breakthrough pain. A large number of routes are available for opioid administration. Opiods may however be associated with side effects due to systemic effects and hallucination.

Oral preparations: Typically, the rescue doses consist of an immediate release preparation that is the same dose as being administered on around the clock basis although the most effective dose remains unknown. Titration of the rescue dose according to the character of breakthrough pain is, therefore, advocated to identify the suitable dose.

Sublingual preparations: This route has limited application due to lack of existing formulations, poor absorption of drugs and inability to deliver high doses that are prevented by swallowing.

Intranasal preparations: Ketamine has been used in the treatment of break through pain (BTP) in chronic patients. In such patients, 10-50 mg of ketamine has been administered through intranasal administration in incremental 10 mg doses, every 90 seconds. The effect of that intranasal administration of ketamine was that there was a lower BTP in patients that received intranasal ketamine as opposed to placebo.

Rectal preparation: Rectal administration offers the possible pharmacokinetic advantage of bypassing first pass metabolism by direct ally entering the systemic circulation via the lower rectal veins. But there is no clear demarcation between portal and systemic drainage and this may render proportion of drug absorbed through portal system difficult to predict. Therefore, a considerable difference in bio-availability of rectally administered morphine has been observed in between individuals.

Transmucosal route: Both oral transmucosal and nasal formulations of fentanyl have become available and studied recently for relief of breakthrough pain. The efficacy of oral fentanyl was compared with morphine sulfate immediate release oral form and it was found that pain relief was earlier and quantitatively better with former. The dose of oral fentanyl used varied from 200-1600 μg. Nasal fentanyl spray 20 ug was also found to be better than oral morphine to relieve breakthrough pain.

Subcutaneous and intravenous route: Parenteral route is best for immediate pain relief. Subcutaneous route is equally efficacious although onset is slower than intravenous route. Previous studies have mainly studied the benefits of morphine sulfate immediate release (MSIR) and fentanyl citrate for the management of breakthrough pain. In one study, oral transmucosal fentanyl citrate (OTFC) was used and pain relief (PR) was measured at 15, 30, 60 minutes post intake. The dose of OTFC varied from 200-1600 μg, the exact dose being decided during the drug titration phase. By exploratory analysis, it was concluded that OTFC provided earlier and better PR than MSIR that was being used by the patients before they entered into this study.

However, one study directly compared the effect of MSIR versus OTFC and concluded that pain intensity, pain relief and global performance of medication scores were significantly better for OTFC.

Nasal fentanyl was used in one study (20 ug) to treat breakthrough pain. It was concluded that 75% patients had better or same pain relief as compared to MSIR that they were using earlier. 33% patients had pain relief within 5 minutes and 75% patients said that they would continue to take nasal fentanyl in preference to MSIR.

Adjuvant Preparations: The regular uses of antidepressants, antiarrhythmics and anticonvulsants have been used to treat pain refractory to opioids and particularly neuropathic pain.

Miscellaneous: Spasmolytics like octreotide are used to treat colicky pain and drugs like bisphosphonates are used to treat metastatic bone disease.

Non-pharmacological methods: Physiatric techniques like physical therapy or use of orthotics are useful in musculoskeletal pain; bracing is of value in movement related pain. Psychological techniques are useful in certain patients.

Invasive measures: Anaesthetic approaches useful in treatment of persistent pain are sometimes useful to treat breakthrough pain like chemical neurolysis and epidural catheter infusion of local anaesthetics, opioids, and clonidine.

A percutaneous cordotomy is useful as a last resort to treat refractory incident pain from bone metastasis. Intrathecal phenol block and pituitary ablation have also been used to treat refractory breakthrough pain.

The results of these invasive procedures are often sub-optimal when considering the risk of side effects.

Breakthrough pain has been associated with a reduced likelihood of adequate pain control. Despite the large and variable incidence of this phenomenon due to varied definitions of this type of pain, only a few studies have been conducted to assess and effectively treat breakthrough pain. However the importance of managing breakthrough pain is acknowledged by all. A large number of drugs from various classes and novel methods of administration like nasal and transmucosal buccal route as in case of fentanyl have been used in these studies to manage this type of pain. However, means for treatment are needed with a quick onset of action and optimal duration that matches the characteristics of breakthrough pain.

Complex regional pain syndrome (CRPS) is a chronic pain condition most often affecting one of the limbs (arms, legs, hands, or feet), usually after an injury or trauma to that limb. CRPS is believed to be caused by damage to, or malfunction of, the peripheral and central nervous systems. The central nervous system is composed of the brain and spinal cord, and the peripheral nervous system involves nerve signalling from the brain and spinal cord to the rest of the body. CRPS is characterized by prolonged or excessive pain and mild or dramatic changes in skin colour, temperature, and/or swelling in the affected area.

There are two similar forms, called CRPS-I and CRPS-II, with the same symptoms and treatments. CRPS-II (previously called causalgia) is the term used for patients with confirmed nerve injuries. Individuals without confirmed nerve injury are classified as having CRPS-I (previously called reflex sympathetic dystrophy syndrome). Some research has identified evidence of nerve injury in CRPS-I, so the validity of the two different forms is being investigated.

CRPS symptoms vary in severity and duration. Studies of the incidence and prevalence of the disease show that most cases are mild and individuals recover gradually with time. In more severe cases, individuals may not recover and may have long-term disability.

Neuropathic pain relates to lesions of the peripheral or central nervous pathways that result in a loss or impairment of pain sensation. Paradoxically, damage to or dysfunction of these pathways can produce pain. For example, damage to peripheral nerves, as occurs in diabetic neuropathy, or to primary afferents, as in herpes zoster, can result in pain that is referred to the body region innervated by the damaged nerves. Although neuropathic pain can be acute in nature, in most patients the pain is persistent (or “refractory”). For a review of neuropathic pain refer to Epidemiology of Refractory Neuropathic Pain (Taylor, Pain Practice, Volume 6, Issue 1, 2006 22-26)

Patients with chronic neuropathic pain are seen most often in clinical practice. It consists of a number of different disease-specific indications, each of which can have differing definitions and cutoffs. It is difficult to estimate precisely the prevalence and incidence of neuropathic pain. The burden of neuropathic pain on patients and healthcare systems appears to be potentially large, with an estimated prevalence of 1.5% (approximately 4 million US patients). Patients with neuropathic pain experience a poor health-related quality of life and consume a high level of healthcare resources and costs.

Oral administration of ketamine has been used for the treatment of chronic pain, but with poor success. As disclosed in Blonk et al. (Use of oral ketamine in chronic pain management: A review; European Journal of Pain, 2009), there was no consistent dose—response relationship observed over multiple studies carried out attempting to use oral administration.

Intranasal administration of Ketamine has also been attempted in treating neuropathic pain (Huge et al., Effects of low-dose intranasal (S)-ketamine in patients with neuropathic pain; European Journal of Pain, 2009). As disclosed therein, the PK profiles reveal sub-optimal kinetics of maximal plasma levels of ketamine. Furthermore, higher doses of intranasally administered ketamine showed poor dose-response properties in addition to increased levels of Norketamine, indicating that at higher doses most of the Ketamine is in fact ingested. Side effects were also higher than expected when administering intranasally.

There is a need for pharmaceutical therapies that can be used to treat patients with the above mentioned disorders, including patients who do not respond to currently available therapies, as well as for pharmaceutical therapies that improve the efficacy of currently available treatment regimes.

Pharmacological strategies that have rapid onset of pain relief/treatment within a short time and that are sustained would therefore have an enormous impact on the quality of life (QoL) and on public health.

Accordingly, an object of the invention is to provide methods and compositions for the treatment of acute or chronic pain which provide rapid and effective control of pain without the harmful side effects associated with traditional analgesics, such as respiratory depression, disturbed sleep patterns, diminished appetite, seizures, and psychological and/or physical dependency.

### SUMMARY OF THE INVENTION

In light of the prior art the technical problem underlying the invention was the provision of alternative or improved means for treating pain. This problem is solved by the features of the independent claims. Preferred embodiments of the present invention are provided by the dependent claims.

Therefore, an object of the invention is to provide a pharmaceutical composition comprising S-Ketamine, salts and/or derivatives thereof for use as a medicament in the treatment of pain, characterised in that said treatment comprises oral transmucosal administration of said pharmaceutical composition to a subject in need of said treatment.

Methods and compositions are provided for use in the treatment of chronic pain, acute pain, break-through cancer pain (BTCP), complex regional pain syndrome (CRPS), refractory cancer pain, neuropathic pain, post traumatic syndrome (PTSD) and/or ischaematic limb pain. More specifically, the invention demonstrates that oral transmucosal administration of S-ketamine is effective to prophylaxis, prevent, reduce, attenuate, eliminate and/or eliminate the symptoms of pain.

In one embodiment the medical use of the pharmaceutical composition according to the present invention is characterised by transbuccal administration. The preferred buccal route of drug delivery provides the direct access to the systemic circulation through the jugular vein bypassing the first pass hepatic metabolism leading to high bioavailability. Other advantages such as excellent accessibility, low enzymatic activity, suitability for drugs or excipients that mildly and reversibly damage or irritate the mucosa, painless administration, easy withdrawal, facility to include permeation enhancer/enzyme inhibitor or pH modifier in the formulation, versatility in designing as multidirectional or unidirectional release system for local or systemic action.

The advantages of buccal delivery systems of the present invention relate to:

1. The oral mucosa has a rich blood supply. Drugs are absorbed from the oral cavity through the oral mucosa, and transported through the deep lingual or facial vein, internal jugular vein and braciocephalic vein into the systemic circulation.
2. Buccal administration, the drug gains direct entry into the systemic circulation thereby bypassing the first pass effect. Contact with the digestive fluids of gastrointestinal tract is avoided which might be unsuitable for stability of many drugs. In addition, the rate of drug absorption is not influenced by food or gastric emptying rate.
3. The area of buccal membrane is sufficiently large to allow a delivery system to be placed at different occasions, additionally; there are two areas of buccal membranes per mouth, which would allow buccal drug delivery systems to be placed, alternatively on the left and right buccal membranes.
4. Buccal patches show improved accessibility to the membranes that line the oral cavity, which makes application painless.
5. Patients can control the period of administration or terminate delivery in case of emergencies. The buccal drug delivery systems easily administered into the buccal cavity. The novel buccal dosage forms exhibits better patient compliance than previously achieved.
6. The drug becomes systematically available by direct uptake through the mucosa. The result is nearly immediate onset of action without passing the liver (no first pass effect) and less metabolite will be generated.
7. The buccal delivery system allows for a distinct dose reduction and causes fewer side effects in contrast to the typical oral application.
8. The buccal delivery systems preferably exhibit muco-adhesive properties upon contact with saliva, resulting in secure adhesion to the application site.
9. The platform can be designed to either dissolve or to remain in its original form and lose adhesion after a certain period of time. The second option is intended to be removed from the oral cavity upon lose of adhesion.
10. The components used are biocompatible and non-toxic hence providing completely safe carrier systems.

In one embodiment the medical use of the pharmaceutical composition according to the present invention is characterised by sublingual administration.

In one embodiment the medical use of the pharmaceutical composition according to the present invention is characterised by administration of oral dry powder, preferably to the oral cavity.

### Fast Oral Transmucosal Formulation:

In one embodiment the medical use of the pharmaceutical composition according to the present invention is characterised by administration of the composition as a fast oral transmucosal (FOT) composition.

The FOT matrix designed for the present invention relates in a preferred embodiment to a combined Mucoadhesive system (preferably a mucoadhesive patch/tablet). The Mucoadhesive system comprises preferably of an orodispersible matrix with S-ketamine. Such an orodispersible matrix may be prepared according to the ODT formulation described herein.

The FOT composition is preferably suitable for uni- or bidirectional release of the active agent S-ketamine, preferably to the buccal mucosa and to the cavity mucosa. In bi-directional release, the two layers are attached to an inert excipient, which is present in between the two outer layers, forming a three- (or more-) layered FOT composition. A water impermeable coating may be present on one or more layers.

Such FOT compositions may also be provided as an orodispersible film, as described herein.

In one embodiment of the invention the composition is not a lollipop, which can lead to undesired ingestion of a significant portion of the active substance.

### Orodispersible Tablet Formulation:

In one embodiment the medical use of the pharmaceutical composition according to the present invention is characterised by administration of the composition as an orodispersible tablet (ODT).

ODT preferably include a taste masking agent and are manufactured by passing through a sieve to ensure the better mixing. Microcristalline Cellulose is preferred as a direct compressible vehicle. Super disintegrants such as Sodium Starch Glycolate, Crospovidone and/or Croscarmellose Sodium are preferred. The ODT preferably comprise of Talc, Magnesium stearate, Aspartame, Microcrystalline cellulose, Sodium starch glycolate and/or Lactose.

The ODT formulation of the invention therefore preferably comprises active agent, one or more excipients, one or more disintegrants and/or swelling agent, optionally one or more sweeteners, one or more lubricants and optionally one or more fillers.

The active agent relates to S-Ketamine, its salts and/or derivatives as described herein.

A preferred excipient is lactose. Excipients are generally a pharmacologically inactive substance formulated with the active ingredient of a medication. Excipient is used to bulk up formulation to allow convenient and accurate dispensation of a drug substance when producing a dosage form. An excipient example is a binder, which holds the ingredients in a tablet together. Binders may be saccharides and their derivatives, such as disaccharides, sucrose, lactose, polysaccharides and their derivatives such as starches, cellulose or modified cellulose such as microcrystalline cellulose and cellulose ethers such as hydroxypropyl cellulose, sugar alcohols such as xylitol, sorbitol or maltitol, protein, gelatin or synthetic polymers, such as polyvinylpyrrolidone (PVP) or polyethylene glycol (PEG).

A preferred disintegrant is sodium starch glycolate, which is the sodium salt of carboxymethyl ether. Alternative starch glycolates may be of rice, potato, wheat or corn origin. Sodium starch glycoate is a white to off-white, tasteless, odorless, relatively free flowing powder. The disintegrant, especially sodium starch glycolate, absorbs water rapidly, resulting preferably in swelling which leads to rapid disintegration of tablets and granules.

The swelling agent relates preferably to microcrystalline cellulose and enables degradation of the formulation and release of the active agent. Swelling agents are hydrophilic crosslinked polymers, which swell from preferably 10 to 1,000 times their own weight when placed in an aqueous medium. Depending on their swelling properties, these materials have been exploited in different classes of materials in pharmaceutical industries, i.e. swellable matrices, as superdisintegrants and/or swelling devices.

Without a suitable disintegrant or swelling agent, tablets may not dissolve appropriately and may effect the amount of active ingredient absorbed, thereby decreasing effectiveness

The sweetener is preferably aspartame, but could be any other sweetener.

The lubricant is preferably magnesium stearate.

The filler is preferably Talc.

The ODT formulation of the invention therefore preferably comprises the components in the following relative ratios (with respect to mass):

Active agent 50-150: excipient 50-200: disintegrant and/or swelling agent 10-200: sweetener 0-20: lubricant 0-10: filler 0-50.

In a preferred embodiment the ODT formulation of the invention therefore preferably comprises the components in the following relative ratios (with respect to mass):

Active agent 80-120: excipient 100-150: disintegrant 5-20: swelling agent 80-200: sweetener 5-15: lubricant 1-5: filler 5-20.

Preferred embodiments are provided in the examples disclosed herein.

### Sustained Release Formulation:

In one embodiment the medical use of the pharmaceutical composition according to the present invention is characterised by administration of the composition as a sustained release (SR) composition.

SR compositions preferably comprise of active substance, Microcrystalline cellulose and Magnesium Stearate, and optionally PEG. Further optional components relate to additional adjuvants (such as swelling agents), preferably capable of unlimited swelling, for example cellulose compounds, such as, but not limited to methylcellulose, cellulose gum, hydroxylpropyl cellulose and hydroxypropyl-methyl cellulose.

The SR formulation of the invention therefore preferably comprises active agent, one or more swelling agents, one or more lubricants and optionally one or more swelling controllers.

The active agent relates to S-Ketamine, its salts and/or derivatives as described herein.

The swelling agent relates preferably to microcrystalline cellulose and enables degradation of the formulation and release of the active agent. Swelling agents are hydrophilic crosslinked polymers, which swell from preferably 10 to 1,000 times their own weight when placed in an aqueous medium. Depending on their swelling properties, these materials have been exploited in different classes of materials in pharmaceutical industries, i.e. swellable matrices, as superdisintegrants and/or swelling devices.

Examples of adjuvants (swelling agents) capable of unlimited swelling are known as cellulose compounds such as, but not limited to methylcellulose, cellulose gum, hydroxylpropyl cellulose, carboxymethyl cellulose and hydroxypropyl-methyl cellulose.

The lubricant is preferably magnesium stearate.

An example of a preferred swelling controller is PEG, with potentially different molecular weights, preferably PEG 35000.

The SR formulation of the invention therefore preferably comprises the components in the following relative ratios (with respect to mass):

Active agent 50-150: swelling agent 10-200: lubricant 1-100: swelling controller 0-10.

In a preferred embodiment the SR formulation of the invention therefore preferably comprises the components in the following relative ratios (with respect to mass):

Active agent 80-120: swelling agent 20-100: lubricant 2-50: swelling controller 2-8.

Preferred embodiments are provided in the examples disclosed herein.

The sustained release formulation of S-ketamine of the present invention can be present in the form of conventional formulation such as tablets or capsules (single unit drug dosage forms). It can also be a multicompartment form, or a part thereof, and, for example, be filled into a capsule. The multicompartment form means dividing the total dose into several small units (microforms such as microcapsules, pellets and microtablets; small microunits, obtained by various preparation processes, e.g., coacervation, extrusion, compression, tabletting.

### Orodispersible Film Formulation:

In one embodiment the medical use of the pharmaceutical composition according to the present invention is characterised by administration of the composition as an orodispersible film (ODF).

The ODF compositions comprise preferably granular hydroxypropyl starch, Hydroxypropyl methyl cellulose, an alcohol, Propylene glycol, Maltodextrin and/or a flavouring agent (such as Menthol) and preferably Distilled Water to make the composition to 100% of desired weight. Also preferred components are polyvinyl alcohol, polyvinyl pyrrolidone, maltodextrin, microcrystalline cellulose, Hydroxypropyl methyl cellulose, modified starch, chitosan, gums and/or blends of these polymers.

The preferred oral film technology represents an innovative form of medication with respect to the present invention. OFT offers advantages to patients and combines the convenience of a liquid with the stability and dosing accuracy of a tablet. The drug can be uni-directionally released to the buccal mucosa, both for local or systemic uptake, or to the oral cavity for local action.

The ODF formulation of the invention therefore preferably comprises active agent, one or more modified starches suitable for film coating, one or more alcohols, one or more pharmaceutically accepted solvents, one or more binders, one or more flavouring agents, and preferably water.

The active agent relates to S-Ketamine, its salts and/or derivatives as described herein. The active agent applied in the film could be 1 to 1000, more preferably 10 to 500, more preferably 50 to 150 mg/4 cm² of the film.

Preferred modified starches suitable for film coating relate to Lycoat NG73 (granular hydroxypropyl starch), hydroxypropyl methyl cellulose or other modified starch.

The alcohol is preferably a short chain alcohol such as ethanol. Pharmaceutically accepted solvents are known in the art. Preferred is propylene glycol.

As binder Maltodextrin is preferred. Maltodextrin is an oligosaccharide. It is produced from starch by partial hydrolysis and is usually found as a white hygroscopic spray-dried powder. Maltodextrin is easily digestible, being absorbed as rapidly as glucose, and might be either moderately sweet or almost flavourless. As alternative binders other excipients are mentioned herein could be applied.

The flavouring agent is preferably menthol, but could be any other flavour.

The ODF formulation of the invention therefore preferably comprises the components in the following relative percentages (with respect to mass; active agent is not included in these amounts but is added to the film as described herein):

modified starch 2-30: alcohol 0-20: solvent 5-20: binder 0-5: flavouring agent 0-5: water to fill the remaining up to 100.

In a preferred embodiment the ODT formulation of the invention therefore preferably comprises the components in the following percentages (with respect to mass; active agent is not included in these amounts but is added to the film as described herein):

modified starch 4-20: alcohol 5-15: solvent 5-10: binder 1-3: flavouring agent 0.2-1: water to fill the remaining up to 100.

Preferred embodiments are provided in the examples disclosed herein.

### Orodispersible Granule Formulation:

In one embodiment the medical use of the pharmaceutical composition according to the present invention is characterised by administration of the composition as orodispersible granules (micro-pellets).

In a preferred embodiment the pharmaceutical composition for use as a medicament according to the present invention is characterised in that the S-ketamine derivative is nor-S-Ketamine, S-Dehydronorketamine or (S,S)-6-Hydroxynorketamine.

In a preferred embodiment the pharmaceutical composition for use as a medicament according to the present invention is characterised in that the S-ketamine salt is S-Ketamine hydrochloride.

In a preferred embodiment the pharmaceutical composition for use as a medicament according to the present invention is characterised in that the S-ketamine salt is a salt of an organic acid, preferably selected from an acetic, trifluoroacetic, propionic, succinic, glycolic, stearic, lactic, malic, tartaric, citric, ascorbic, or amino acid salt.

In further embodiments of the invention the pharmaceutical composition for use as a medicament according to the present invention is characterised in that the S-ketamine amino acid salt is arginate, asparginate, or glutamate.

In further embodiments of the present invention the pain to be treated is chronic pain, such as chronic break-through pain (BTP), break through pain (BTP), complex regional pain syndrome (CRPS), refractory cancer pain, neuropathic pain, post traumatic syndrome pain (PTSD), ischaematic limb pain and/or acute pain.

The pharmaceutical composition for use as a medicament according to the present invention is characterised in that the composition is administered at a dosage sufficient to prevent (prophylaxis), reduce, attenuate, eliminate and/or therapeutically treat the symptoms of said pain.

In one embodiment the medical use of the pharmaceutical composition according to the present invention is characterised by administration of a single dose of said composition.

In one embodiment the medical use of the pharmaceutical composition according to the present invention is characterised by administration of a multiple dose of said composition.

In one embodiment the medical use of the pharmaceutical composition according to the present invention is characterised by administration of the composition at a dose of between about 0.05 mg/kg BW per day to about 6 mg/kg BW per day.

In other embodiments the invention encompasses a dose of between 0.01 mg/kg BW to 10 mg/kg BW per day, preferably 0.1 mg/kg BW to 5 mg/kg BW per day, preferably 0.5 mg/kg BW to 4 mg/kg BW, more preferably 0.9 mg/kg BW to 3 mg/kg BW per day.

In one embodiment the medical use of the pharmaceutical composition according to the present invention is characterised in that the OFT composition is administered at a single dose of between 10 to 200 mg of S-Ketamine.

In one embodiment the medical use of the pharmaceutical composition according to the present invention is characterised in that the OFT composition is administered at a single dose of between 20 to 150 mg of S-Ketamine.

In one embodiment the medical use of the pharmaceutical composition according to the present invention is characterised in that the OFT composition is administered at a single dose of between 40 to 120 mg of S-Ketamine.

In one embodiment the medical use of the pharmaceutical composition according to the present invention is characterised in that the OFT composition is administered at a single dose of 100 mg of S-Ketamine.

In one embodiment the medical use of the pharmaceutical composition according to the present invention is characterised in that the SR composition is administered at a single dose of between 100 to 500 mg of S-Ketamine.

In one embodiment the medical use of the pharmaceutical composition according to the present invention is characterised in that the SR composition is administered at a single dose of between 200 to 400 mg, preferably about 300 mg, of S-Ketamine.

In one embodiment the medical use of the pharmaceutical composition according to the present invention is characterised in that the SR composition is administered at a single dose providing between 10 to 50 mg of S-Ketamine per hour, preferably 25 mg of S-Ketamine, for 8 to 16 hours, preferably for about 12 hours.

In a further aspect of the invention the medical use of the pharmaceutical composition according to the present invention is characterised in that the composition is administered in combination with opioid therapy in cancer patients with pain.

In one embodiment the medical use of the pharmaceutical composition according to the present invention is characterised by administration of a pharmaceutically effective dose of a second agent, preferably selected from the group consisting of a pharmaceutical NMDA receptor antagonist, analgesic drug, narcotic analgesic opioid, a non-steroidal anti-inflammatory analgesic (NSAIA), antidepressant, neuroleptic agent, anticonvulsant, a mood stabilizer, an antipsychotic agent, anticancer agent and benzodiazepine.

A further aspect of the invention relates to a kit for administration of a medicament, comprising in close confinement at least a) S-ketamine, salts and/or derivatives thereof and b) a pharmaceutical carrier suitable for oral transmucosal administration, and optionally c) a second component according to the preceding claim.

The invention therefore relates to a pharmaceutical composition for use as a medicament according to the present invention, comprising S-ketamine, salts and/or derivative thereof, and one or more pharmaceutically acceptable oral transmucosal carrier substances.

In a preferred embodiment the pharmaceutical composition for use as a medicament according to the present invention, comprises S-ketamine, salts and/or derivative thereof, and one or more pharmaceutically acceptable oral transbuccal carrier substances. The buccal systems preferably exhibit muco-adhesive properties upon contact with saliva, resulting in secure adhesion to the application site. The platform can be designed to either dissolve or to remain in its origin form and los adhesion after a certain amount of time. The second option is intended to be removed from the oral cavity upon loss of adhesion.

In a preferred embodiment the pharmaceutical composition for use as a medicament according to the present invention, comprises S-ketamine, salts and/or derivative thereof, and one or more pharmaceutically acceptable oral sublingual carrier substances.

In a preferred embodiment the pharmaceutical composition for use as a medicament according to the present invention, comprises S-ketamine, salts and/or derivative thereof, and one or more pharmaceutically acceptable carrier substances for an oral dry powder.

The invention therefore also relates to a method of treating a human subject for pain comprising oral transmucosal administration of a pharmaceutical composition comprising S-Ketamine, salts and/or derivatives thereof as described herein to a subject in need of said treatment.

The invention also encompasses a method of treating a human patient for acute and chronic pain comprising intranasal, transdermal, spray inhalation, rectal, intravenous, topical and/or local administration of a composition comprising S-ketamine, its salt, and/or derivative to said patient at a dosage sufficient to prophylaxis, prevention, reduce, attenuate, eliminate and/or therapy the symptoms of said acute, chronic break-through pain (BTCP), complex regional syndrome (CRPS), refractory cancer pain, neuropathic pain, post traumatic syndrome (PTSD) and/or ischaematic limb pain.

The invention therefore encompasses a device for patient self-administration of S-ketamine, its salt, and/or derivative comprising a nasal spray or powder inhaler containing an aerosol spray formulation of S-ketamine, its salt, and/or derivative and a pharmaceutically acceptable dispersant, wherein the device is metered to disperse an amount of the aerosol formulation by forming a spray that contains a dose of S-ketamine effective to reduce or eliminate the symptoms of pain.

The invention therefore encompasses a kit for administration of a medicament comprising in close confinement at least a) S-ketamine, salts and/or derivatives thereof and b) a device for self-administration of an intranasal formulation, and optionally c) a second agent selected from the group consisting of a pharmaceutical NMDA receptor antagonist, analgesic drug, narcotic analgesic opioid, a non-steroidal anti-inflammatory analgesic (NSAIA), antidepressant, neuroleptic agent, anticonvulsant, a mood stabilizer, an antipsychotic agent, anticancer agent and benzodiazepine.

The invention encompasses a kit for administration of a medicament comprising in close confinement at least a) S-ketamine, salts and/or derivatives thereof and b) a device for self-administration of a transdermal formulation such as a transdermal patch, and optionally c) a second agent selected from the group consisting of a pharmaceutical NMDA receptor antagonist, analgesic drug, narcotic analgesic opioid, a non-steroidal anti-inflammatory analgesic (NSAIA), antidepressant, neuroleptic agent, anticonvulsant, a mood stabilizer, an antipsychotic agent, anticancer agent and benzodiazepine.

The invention encompasses a pharmaceutical composition comprising S-Ketamine, salts and/or derivatives thereof for use as a neuro-protective medicament in subjects with brain and/or spinal cord injuries, characterised in that said treatment comprises oral transmucosal administration of said pharmaceutical composition to a subject in need of said treatment.

The invention encompasses a pharmaceutical composition comprising S-Ketamine, salts and/or derivatives thereof for use as a medicament in treatment of depression and/or CNS-disorders, characterised in that said treatment comprises oral transmucosal administration of said pharmaceutical composition to a subject in need of said treatment.

The invention also encompasses a method of treating a human patient for acute or chronic pain comprising orally administering a composition comprising nor S-ketamine, S-Dehydronorketamine, and/or (S,S)-6-Hydroxynorketamine to said patient at a dosage sufficient to treat the symptoms of said acute, chronic break-through pain (BTCP), complex regional syndrome (CRPS), refractory cancer pain, neuropathic pain, post traumatic syndrome (PTSD) and/or ischaematic limb pain.

### DETAILED DESCRIPTION OF THE INVENTION

### Definition of Terms

It should be understood that the purposes of the present invention, the following terms have the following meanings:

The term “effective analgesia” is defined for purposes of the present invention as a satisfactory reduction in or elimination of pain, along with the production of tolerable level of side effects, as determined by the human patient.

The term “effective pain management” is defined for the purposes of the present invention as the objective evaluation or opinion of a human patient response (pain experienced versus side effects) to analgesic treatment by a physician as well as subjective evaluation of therapeutic treatment by the patient undergoing such treatment. The skilled artisan will understand that effective analgesia will vary widely according to many factors, including individual patient variable.

The term “pain” is defined for the purposes of the present invention as the unpleasant sensation localized to a part of the body. It is often described in terms of a penetrating or tissue-destructive process (e.g., stabbing, burning, twisting, tearing, squeezing) and/or a bodily or emotional reaction (e.g., terrifying, nauseating, sickening). Furthermore, any pain of moderate or higher intensity as accompanied by anxiety and the urge to escape or terminate the feeling. These properties illustrate the duality of pain: it is both sensation and emotion.

The term “acute pain” is defined for the purposes of the present invention as pain characteristically associated with behavioural arousal and a stress response consisting of increased blood pressure, heard rate, pupil diameter, and plasma cortisol levels. In addition, local muscle contraction (e.g., limb flexion, abdominal wall rigidity) is often present.

The term “chronic pain” is defined for the purposes of the present patent that there are several factors that can cause, perpetuate, or exacerbate chronic pain. Firstly of course, the patient may simply have a disease that is characteristically painful for which there is presently no cure. Arthritis, cancer, migraine, headaches, fibromyalgia, and diabetic neuropathy are examples of this. Secondly, there may be secondary perpetuating factors that are initiated by disease and persist after that disease has resolved. Examples include damaged sensory nerves, sympathetic efferent activity, and painful reflex muscle contraction. Finally, a variety of psychological conditions can exacerbate or even cause pain.

The term “break-through pain (BTP)” is defined for the purposes of the present invention as exacerbations of preferably significant and/or severe pain on a background of otherwise controlled pain. Such “flare ups” of pain are known as breakthrough pain, as the pain “breaks through” the regular pain medication. Characteristics that can further define breakthrough pain include its relation to the fixed dose of opioid medication, temporal features, precipitating events and its predictability. In the United Kingdom, this term is used as a sign of end of dose failure during dose titration for pain management. Some experts have advocated the use of broader terms like episodic pain or transient pain in place of breakthrough pain, whereas some have listed the types of breakthrough pain depending on its predictability and precipitating factors. Following are the types of breakthrough pain:
Idiopathic
Incidental
End of Dose

Idiopathic pain (Spontaneous): Stimulus independent i.e no obvious precipitating factor. Pain comes on without warning and has no precipitating stimulus. Sudden, sharp, and often marked by a disabling crescendo, idiopathic pain is common in neuropathic pain condition.

Incidental pain: Incidental pain has an identifiable cause. The cause can be volitional, as in pain caused when the patient initiates movement such as walking, or nonvolitional, as in the type of pain that can occur during bladder spasm after voiding. The most common type of BTP in cancer patients is incident pain related to bone metastases, but cancer patients are also subject to sudden paroxysmal pain associated with neuropathic origins.

End of dose pain: It results when the dose of drug drops below the analgesic level. End of dose pain occurs with greater frequency at the end of dosing interval of around the clock opioid medication.

The term “refractory cancer” is defined for the purposes of the present invention as a malignancy for which surgery is ineffective, which is either initially unresponsive to chemo- or radiation therapy, or which becomes unresponsive over time.

The term “refractory cancer pain” is defined for the purposes of the present invention as pain has persisted over time despite an adequate trial of analgesic therapies, therapeutic interventions, and non-pharmacological approaches including the recognition and response to suffering. Refractory cancer pain can be defined for the purposes of the present invention as pain, which can be acute in nature, in most patients the pain is persistent (or “refractory”).

The term “complex regional pain syndrome” is defined for the purposes of the present invention as a chronic pain condition most often affecting one of the limbs (arms, legs, hands, or feet), usually after an injury or trauma to that limb. CRPS is believed to be caused by damage to, or malfunction of, the peripheral and central nervous systems. The central nervous system is composed of the brain and spinal cord, and the peripheral nervous system involves nerve signaling from the brain and spinal cord to the rest of the body. CRPS is characterized by prolonged or excessive pain and mild or dramatic changes in skin colour, temperature, and/or swelling in the affected area.

There are two similar forms, called CRPS-I and CRPS-II, with the same symptoms and treatments. CRPS-II (previously called causalgia) is the term used for patients with confirmed nerve injuries. Individuals without confirmed nerve injury are classified as having CRPS-I (previously called reflex sympathetic dystrophy syndrome). Some research has identified evidence of nerve injury in CRPS-I, so the validity of the two different forms is being investigated.

CRPS symptoms vary in severity and duration. Studies of the incidence and prevalence of the disease show that most cases are mild and individuals recover gradually with time. In more severe cases, individuals may not recover and may have long-term disability.

The prevalence of chronic pain in France is more than 31%; of these, 20% have the characteristics of neuropathic pain (ie, some 6% of the total population). The term “neuropathic pain”, is defined for the purposes of the present invention as a pain that can be acute in nature, in most patients the pain is persistent (or “refractory”). Patients with chronic neuropathic pain are seen most often in clinical practice. It consists of a number of different disease-specific indications, each of which can have differing definitions and cutoffs. It is difficult to estimate precisely the prevalence and incidence of neuropathic pain. The burden of neuropathic pain on patients and healthcare systems appears to be potentially large, with an estimated prevalence of 1.5% (approximately 4 million US patients). Patients with neuropathic pain experience a poor health-related quality of life and consume a high level of healthcare resources and costs.

The term “post-traumatic syndrome PTSD” is defined for the purposes of the present invention as a potentially debilitating anxiety disorder triggered by exposure to a traumatic experience such as an interpersonal event like physical or sexual assault, exposure to disaster or accidents, combat or witnessing a traumatic event. There are three main clusters of symptoms: firstly, those related to re-experiencing the event; secondly, those related to avoidance and arousal; and thirdly, the distress and impairment caused by the first two symptom clusters. Both psychological therapy and pharmacotherapy have been used to treat PTSD and guidelines suggest that a combination of both may mean people recover from PTSD more effectively.

The term “Ischaemic limb pain” is defined for the purposes of the present invention as pain caused by acute limb ischemia defined as a sudden decrease in limb perfusion that causes a potential threat to limb viability (manifested by ischemic rest pain, ischemic ulcers, and/or gangrene) in patients who present within two weeks of the acute event (if >2 weeks, it is considered chronic ischaemia). Chronic critical limb ischemia is manifested by pain at rest, non healing wounds and gangrene. Ischemic rest pain is typically described as a burning pain in the arch or distal foot that occurs while the patient is recumbent but is relieved when the patient returns to a position in which the feet are dependent.

The term “mental disorder” or “mental illness” is defined for the purposes of the present invention as a medical condition that disrupt a person's thinking, feeling, mood, ability to relate to others and daily functioning. Mental illnesses are medical conditions that often result in a diminished capacity for coping with the ordinary demands of life. Serious mental illnesses include major depression, schizophrenia, bipolar disorder, obsessive compulsive disorder (OCD), panic disorder, post traumatic stress disorder (PTSD) and borderline personality disorder.

The term “oral transmucosal” administration or delivery is defined for the purposes of the present invention as delivery of active substance systemically and/or locally across a mucous membrane in the oral cavity, preferably via buccal or sublingual mucosa. Buccal delivery refers to the drug release which can occur when a dosage form is placed in the outer vestibule between the buccal mucosa and gingival.

The term “buccal dosage form” is defined for the purposes of the present invention as the buccal dosage forms including buccal adhesive tablets, patches, films, semisolids (ointments and gels) and powders:

### A. Buccal Mucoadhesive Tablets

Buccal mucoadhesive tablets are preferably dry dosage forms that have to be moistened prior to placing in contact with buccal mucosa. Example: a double or three-layered tablet, consisting of mucoadhesive matrix layer, preferably of hydroxy propyl cellulose and/or polyacrylic acid, optionally an inner inert layer of preferably cocoa butter, which may be preferably coated, whereby the adhesive layer may contain S-ketamine and preferably a penetration enhancer (such as sodium glycocholate). An additional orodisperible matrix may also be provided, whereby the inert layer is maintained between the adhesive and orodispersible layers, thereby enabling a three-layer tablet, capable of bidirectional release of active substance.

### B. Patches and Films

Buccal patches may be a single film or comprise of two laminates, with an aqueous solution of the adhesive polymer being cast onto an impermeable backing sheet, which is then cut into the required oval shape. Example: A mucosal adhesive film, similar to such known as “Zilactin”—consisting of an alcoholic solution of hydroxy propyl cellulose and three organic acids. The film which is applied to the oral mucosal can be retained in place for preferably 12 hours or more even when it is challenged with fluids.

### C. Semisolid Preparations (Ointments and Gels)

Bioadhesive gels or ointments generally have less patient acceptability than solid bioadhesive dosage forms, and most of the dosage forms are used only for localized drug therapy within the oral cavity. Example: One of the original oral mucoadhesive delivery systems—“orabase”—consists of finely ground pectin, gelatin and sodium carboxy methyl cellulose dispersed in a poly (ethylene) and a mineral oil gel base, which can be maintained at its site of application for 15-150 minutes.

### D. Powders

An example of a powder relates to hydroxpropyl cellulose and S-Ketamine in powder form, suitable for being sprayed onto the oral mucosa of patient.

The term “oral mucosa” is defined for the purposes of the present invention as the mucous membrane epithelium (and lamina propria) of the mouth. It can be divided into various categories.
Masticatory mucosa, para-keratinized stratified squamous epithelium, found on the dorsum of the tongue, hard palate and attached gingiva.
Lining mucosa, non-keratinized stratified squamous epithelium, found almost everywhere else in the oral cavity.
Buccal mucosa refers to the inside lining of the cheeks and is part of the lining mucosa.
Specialized mucosa, specifically in the regions of the taste buds on the dorsum of the tongue.

The oral mucosa has several functions. Its main purpose is to act as a barrier. It protects the deeper tissues such as fat, muscle, nerve and blood supplies from mechanical insults, such as trauma during chewing, and also prevents the entry of bacteria and some toxic substances into the body. The oral mucosa has an extensive innervation of nerves, which allows the mouth to be very receptive of hot and cold, as well as touch. Taste buds are also located in oral mucosa and are important for recognition of taste.

The major secretion associated with the oral mucosa is saliva, produced by the salivary glands. The major salivary glands secrete most of the saliva via ducts that pass through the oral mucosa. There is a degree of permeability that allows for rapid absorption into the body in certain circumstances e.g. the permeability of the oral mucosa is utilised in the rubbing of orange juice, or another sugary drink when diabetics suffer from a low-blood sugar.

The term “sublingual delivery”, is defined for the purposes of the invention as delivery system consisting of administration through the membrane of the ventral surface of the tongue and the floor of the mouth. They compromise of orally disintegrating or dissolving medications that are administering by being placed under the tongue. Drugs diffuse into the blood through tissues under the tongue.

The mucous membranes (or mucosae or mucosas; singular mucosa) are linings of mostly endodermal origin, covered in epithelium, which are involved in absorption and secretion. They line cavities that are exposed to the external environment and internal organs. They are at several places contiguous with skin: at the nostrils, the lips of the mouth, the eyelids, the ears, the genital area and the anus. The sticky, thick fluid secreted by the mucous membranes and glands is termed mucus. The term mucous membrane refers to where they are found in the body and not every mucous membrane secretes mucus. The glans clitoridis, glans penis (head of the penis), along with the inside of the foreskin and the clitoral hood, are mucous membranes. The urethra is also a mucous membrane. The secreted mucus traps the pathogens in the body, preventing any further activities of diseases.

The term “transdermal drug delivery” is defined for the purposes of the present invention as, relating to, being, or supplying a medication in a form for absorption through the skin into the bloodstream.

The term “local drug delivery” is defined for the purposes of the present invention as relating to, being, or administration of a drug through all areas other than the sublingual and buccal delivery.

The term “fast oral transmucosal (FOT)” is defined for the purposes of the present invention as relating to, being, or administering medication in a form for absorption through all areas of buccal mucosa and the sublingual route into the bloodstream.

The term “orodispersible films (ODF)” is defined for the purposes of the present invention as strips of thin polymeric films, preferably disintegrating or dissolving instantaneously when administered to the oral cavity.

The term “rapid film” is defined for the purposes of the present invention as very thin film which is applied in the mouth. It is based on water soluble polymers. The design can vary from single to multilayer systems.

The term “orodispersible tablets (ODT)” is defined for the purposes of the invention as coated or uncoated tablets intended to be placed in the mouth where they disperse rapidly before being swallowed.

The term “orodispersible granules (micro-pellets)” is defined for the purposes of the patent coated or uncoated particles for immediate or sustained release filled in stick packs or sachets intended to be placed in the mouth where they disperse rapidly before being swallowed.

### DESCRIPTION OF PREFERRED ADVANTAGES THE INVENTION

The present invention is based on the surprising and unexpected discovery that buccal administration of S-ketamine can reduce and/or eliminate symptoms of acute and/or chronic pain in patients suffering from break-through cancer pain, complex regional pain syndrome, refractory cancer pain, neuropathic pain, post-traumatic syndrome, and/or Ischemic limb pain. For example, S-Ketamine-FOT as an analgesic agent has proven surprisingly to be of effect in patients with severe pain who failed to respond to routine pharmacotherapy.

Existing therapies for acute and/or chronic pain in patients suffering from break-through cancer pain, complex regional pain syndrome, refractory cancer pain, neuropathic pain, post-traumatic syndrome, and/or Ischemic limb pain use high doses of anti-inflammatory drugs and opioids resulting in severe side effects and poor quality of life.

More specifically, the existing therapies for break-through-cancer pain require mostly intravenous application of the anti-pain drugs. Due to the unpredicted occurrence and the severity of the pain, it is inconvenient for the patients and time consuming to hospitalize the patients and arrange the intravenous treatment.

The pharmacological management of break-through cancer pain, complex regional pain syndrome, refractory cancer pain, neuropathic pain, post-traumatic syndrome, and/or Ischemic limb pain has been traditionally based on various regimens of opiates and their congeners or NSAIDs.

All opiates have side effects, of which the most dangerous are respiratory and cardiovascular depression associated with excessive sedation. NSAIDs may also induce side effects such as exacerbation of bleeding tendencies and the impairment renal function.

The search of alternative pain control strategies has focused on the N-methyl-D-aspartate (NMDA) receptors and their antagonists, which were recently shown to alleviate somatic and neuropathic pain sensation both animal and human models.

The clinical utility of these agents stems from the high affinity binding of the drugs to NMDA receptors resulting in blockade of NMDA receptors located at the junction where pain is generated by peripheral nociceptive stimuli and thence conveyed to central receptors via A and C sensory fibres. From a clinical standpoint, the amounts of conventional pain killers that are needed for effective pain control would be much smaller.

Based on the preclinical and preliminary studies with Ketamine, the inventors postulated that S-ketamine as NMDA receptor antagonist may reduce and/or eliminate the acute and/or chronic pain in patients resulting in mild side effects.

### NMDA Receptor (N-Methyl-D-Aspartate Receptor) Antagonists:

The NMDA receptor antagonists of the instant invention are agents that block NMDA in the brain and spinal cord, which increases the activity of another receptor, AMPA, and this boost in AMPA activity is crucial for ketamine's rapid antidepressant actions. NMDA and AMPA are receptors for the neurotransmitter glutamate. The glutamate system has been implicated in depression recently.

The compounds of this invention inhibit GABA and may also block serotonin, noradrenaline (norepinephrine) and dopamine in the central nervous system. Though to induce analgesia and amnesia by functionally disrupting the central nervous system through overestimation or induction of a cataleptic state.

In some embodiments, the NMDA receptor antagonist is the stereoisomer (S)-Ketamine ((S)-(+)-Ketamine), S-ketamine hydrochloride, S-ketamine acetate, S-Ketamine sulphate, nor S-ketamine, Ketamine, S-Dehydronorketamine, or (S,S)-6-Hydroxynorketamine.

In more specific embodiments of the invention, the NMDA receptor antagonist is (S)-Ketamine.

In specific embodiments of the invention, the NMDA receptor antagonist is nor (S)-Ketamine.

The NMDA receptor antagonist (S)-Ketamine is known in the art. This and other NMDA receptor antagonists may be synthesised by standard chemical techniques as is well known in the art.

In some embodiments of the invention, the NMDA receptor antagonist includes more than one of the above defined NMDA receptor antagonist.

The NMDA receptor antagonist of the instant invention can exist in different stereoisomeric forms. These compounds can be, for example racemates or optically active forms.

Unless otherwise specified, or clearly indicated by the text, a NMDA receptor antagonist of the instant invention includes the free base or free acid forms of the compound of the invention, if any, as well as any and all pharmaceutically acceptable salt forms of the NMDA receptor antagonist. Such salt forms include derivatives of the NMDA receptor antagonist. Examples of pharmaceutically acceptable salt forms include, but are not limited to, salts derived from mineral, organic and/or metallic salts such as sodium salt, potassium salt, cesium salt and Lithium salt.

As used herein, the compounds of the invention are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention, which upon administration to a recipient, is capable of providing or provides (directly or indirectly) a compound of this invention.

Accordingly, this invention also provides prodrugs of the compounds of the invention, which are derivatives that are designed to enhance biological properties such as oral absorption, clearance, metabolism or compartmental distribution.

### Ketamine (R,S Ketamine):

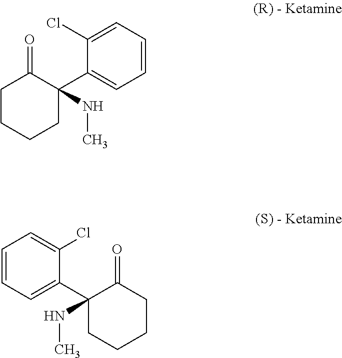 [SMILES: CN[C@@]1(c2ccccc2Cl)CCCCC1=O ; CN[C@]1(c2ccccc2Cl)CCCCC1=O]

Ketamine: dl-2-(o-chloro-phenyl)-2(methylamino)cyclohexanone is as racemate, meaning that both enantiomeres are present in a 50:50 mix.

The liver microsomal enzyme system metabolizes ketamine involving hydroxylation and demethylation, therefore that decrease hepatic blood flow will retard clearance and prolong ketamine effect.

Clearance of ketamine is relatively high at 12-17 ml/kg/minute as a result of a fairly short elimination halftime of about 2.5 hours. Urinary excretion of unchanged drug is about 3-4%, protein binding about 12%. The high lipid solubility of ketamine (ketalar) would have a very large volume of distribution and results in a rapid onset of action. The recovery from the anesthetic effects is properly due to redistribution from the brain to other compartments.

Time to onset following iv bolus (dosage 2 mg/kg) approximately 140 mg/BW is about 30-60 seconds with effect lasting between 10-15 minutes. Complete recovery occurs soon after.

Pharmacology of R,S Ketamine (ketamine):

Ketamine was long thought to act primarily by inhibiting NMDA Receptors. But another NMDA receptor antagonist, MK-801, does not exert the same hypnotic effects. It appears more likely that the hypnotic effects of ketamine are produced by inhibiting hyperpolarization-activated cyclic nucleotide-modulated (HCN1) cation channels, which mediate the “sag” current (Ih) in neurons.

Ketamine is a non-competitive NMDA receptor antagonist. This receptor opens in response to binding of the neurotransmitter glutamate, and blockade of this receptor is believed to mediate the analgesic (reduction of pain) effects at low doses. Evidence for this is reinforced by the fact that naloxone, an opioid antagonist, does not reverse the analgesia. Studies also seem to indicate that ketamine is “use dependent” meaning it only initiates its blocking action once a glutamate binds to the NMDA receptor.

At high, fully anaesthetic level concentrations, ketamine has also been found to bind to opioid mu2 receptors in cultured human neuroplastoma cells without being an agonist on them and sigma receptors. It has also shown to act as a weak D2 receptor partial agonist in rat brain cell homogenates, as well as a dopamine reuptake inhibitor.

### Potential Adverse Effects of R,S Ketamine Described in the Art:

Potential Short term side effects of ketamine are: Increase in heart rate, Slurred speech, Confusion, disorientation, Out-of-body experience, Shifts in perception of reality, Nausea, Sedation, Cardiovascular effects, including hypertension and tachycardia, Respiratory depression, Pleasant mental and/or body high, Increase in energy, Euphoria, Sense of calm and serenity, Meaningful spiritual experiences, Enhanced sense of connection with the world (being or objects), Distortion or loss of sensory perceptions (common), Open and closed-eye visuals (common), Dissociation of mind from body, Analgesia, numbness, Ataxia (loss of motor coordination), Significant change in perception of time, Double-vision.

Potential long term side effects of ketamine use relate to impairments regarding memory. The first large-scale, longitudinal study of ketamine users found that heavy ketamine users had impaired memory by several measures, including verbal, short-term memory and visual memory. However occasional (1-2 times per month) ketamine users and ex-ketamine users were not found to differ from controls in memory, attention and psychological well-being tests. This suggests that occasional use of ketamine does not lead to prolonged harm and that any damage that might occur may be reversible when ketamine use is stopped. The reported short and long-term adverse effects of racemic R,S ketamine have resulted in reduced clinical use.

Preliminary assessment of the experimental studies provide herein has revealed a surprising and beneficial absence of the previously reported side effects.

### Uses of Intravenous R,S Ketamine:

Previously disclosed uses of Ketamine relate to general anaesthesia, usually in combination with a sedative, analgesia (particularly in emergency medicine), sedation in intensive care, treatment of bronchospasm.

It has been shown to be effective in treating depression in patients with bipolar disorder. Ketamine may be used in small doses (0.1-0.5 mg/kg as a local anaesthetic, particularly for the treatment of pain associated with movement and neuropathic pain. Low-dose ketamine is recognized for its potential effectiveness in the treatment of Complex Regional Pain Syndrome (CRPS).

Low-dose ketamine therapy is established as a generally safe procedure. There are two treatment modalities, the first consist of a low dose ketamine infusion of between 25-90 mg per day, over five days. This is called the “awake technique”. The second treatment modality consists of putting the patient in a medically-induced coma and given an extremely high dosage of intravenous R,S ketamine typically between 600-900 mg/day.

### Pharmacokinetics of R,S Ketamine:

Ketamine (ketalar) contains a chiral centre at the C-2 carbon of the cyclohexanone ring, so that two enantiomers exist S-(+)-ketamine and R-(−)ketamine. Consistent with the idea that anesthetics interact specifically with receptors, their differences between the biological activities of the enantiomers with one exhibiting a more rapid onset of action and higher potency. Despite this difference, ketamine (ketalar) is used as a racemate, meaning that both enantiomeres are present in a 50:50 mix.

A number of receptor systems appear to interact with ketamine including the NMSA receptor (N-methyl-D-aspartate), the opioid receptor, adrenergic receptors, muscarinic receptors, as well as voltage-sensitive calcium ion channels. By contrast to barbiturates and benzodiazipines, ketamine (ketalar) does not appear to interact with the GABA receptor system.

Ketamine may be administered by the intravenous, intramuscular, intranasal, oral, and rectal routes. Bioavailability following an intramuscular dose is 93%, intranasal dose 25-50%, and oral dose 20±7%. There are until now no proven significant differences between the pharmacokinetic properties of the S-(+) and R-(−)-isomers.

Peak plasma concentrations have been reported to occur within 1 min following intravenous administration, within 5-10 minutes following intramuscular injection, and 30 minutes after oral administration. Absorption of rectal ketamine in children has been reported to peak at 45 minutes.

Ketamine has high lipid solubility and low plasma protein binding (12%), which facilitates rapid transfer across the blood-brain barrier.

Ketamine readily crosses the placenta and is rapidly distributed into the brain and other highly perfused tissues.

Studies in animals reveal that ketamine is highly concentrated in the lung, body fat, and liver.

The alpha phase of ketamine distribution lasts about 45 minutes, with a half-life of 10-15 minutes. The first phase corresponds clinically to the anesthetic effect of the drug. When administered intravenously, a sensation of dissociation occurs in 15 seconds, and anesthesia occurs within 30 seconds (in 3-4 minutes for IM route).

The anesthetic effects are terminated by a combination of redistribution and hepatic biotransformation to an active metabolite, which is about as active as ketamine in reducing halothane MAC requirements. The beta phase half-life of ketamine is about 2-3 hours. Metabolites are excreted renally (90%) and fecally (5%), with 4% of an administered dose excreted unchanged in urine. Anesthesia lasts 5 to 10 minutes for IV administration and 12-25 minutes for IM administration.

### Absorption, Distribution and Excretion of Ketamine:

Ketamine is eliminated via the kidneys. Animal studies indicate that ketamine hydrochloride is rapidly absorbed after parenteral administration and rapidly distributed to all body tissues. Relatively high concentrations are found in body fat, liver, lung, and brain; lower concentration in heart, skeletal muscle, and blood plasma. Placental transfer has been shown to occur in dogs and monkeys. Placental transfer of ketamine occurred after iv doses to women and the levels of anesthetic in cord blood were equal to, or exceeded, those in plasma within 1.5 min of dosing. The apparent volume of distribution is 3.3 l/kg, and the clearance rate is 1.3 l/min.

### Biological Half-Life Ketamine and S-Ketamine:

Ketamine: T₁₈₂ alpha: 027(0.13)(h); T½ beta: 4.98 (1.56)(h);

Norketamine: T_{1/2}: 5.32 (1.70) (h)

Dehydronorketamine: T_{1/2}: 6.91 (1.71) (h)

The distribution half-life is approximately 7 to 11 minutes.

### Metabolism/Metabolites of Ketamine and S-Ketamine:

Biotransformation of ketamine into multiple metabolites occurs in the liver. The most important pathway invoves N-demethylation by cytochrome p450 to norketamine. Norketamine is then hydroxylated and conjugated to water-soluble compounds that are excreted in urine.

Ketamine is converted to norketamine, 4-,5- and 6-hydroxynorketamines and possibly 4- and 6-hydroxyketamines in hepatic microsomal preparations from rats, rabbits and man. Norketamine is the major metabolite in all species tested.

Biotransformation of ketamine in rhesus monkeys and in man involves oxidative N-demethylation, hydroxylation of the cyclohexanone ring, and dehydration of the hydroxylated metabolites to give the cyclohexanone derivative. 6-Hydroxynorketamine is the major hydroxylated metabolite.

Advantages of S(+)Ketamine compared to R(−)Ketamine:

The administration of S-Ketamine produces reduced spontaneous movements and fewer indications or irregular heartbeat. The therapeutic index of S-Ketamine is 2.5 times larger than that of R-Ketamine. Treatment with S-Ketamine results in a shorter time spent recovering from its effects, for example a shorter waking up time. Anterograde amnesia is less common with S-Ketamine, and concentration capabilities of patients are higher. The “pain killing” and anaesthetic effects are also higher than for R-Ketamine.

The racemate has shown a stronger effect with depressive patients, however, the dream experiences of patients of S-Ketamine seem to be more positive and less troubling when compared to patients receiving the racemate or R-Ketamine.

The increases in blood pressure after administration are comparable between the racemate and S-Ketamine. However, the increase in heart beat frequency is noticeably lower for S-Ketamine when compared to R-Ketamine. Further comparisons between the S-Ketamine and racemate are being carried out with respect to waking up phase, psychomimetic side effects and anaesthetic effect.

### Comparisons Between Intranasal and Oral Transmucosal (S)-Ketamine:

Low-dose intranasal administration of (S)-ketamine rapidly produces adequate plasma concentrations of (S)-ketamine and consecutively sustained concentrations of (S)-norketamine without induction of major remarkable side effects. Ongoing neuropathic pain was significantly and dose-dependently reduced for about 2-3 h.

However, the maximal pain reduction of approximately 30% and 40% was reached 60 min after nasal(S)-ketamine application, which is very slow compared to the FOT administration of the present invention. The examples show that the time course of pain reduction after oral transmucosal administration highly correlates with the respective combined plasma concentrations of (S)-norketamine and (S)-ketamine.

Intranasal drug administration induces a relatively slow (S)-ketamine plasma-peak within 15 min, whereby oral transmucosal administration has provided plasma peaks of 2-3 minutes. Intranasal administration is further hampered by a fast decline below a level of 10 ng/ml within 60 min.

The resulting plasma concentrations of (S)-norketamine, which is the major metabolite of (S)-ketamine synthesized in the liver, exceeded a level of 10 ng/ml between 30 and 180 min after intranasal administration.

As the intranasal route of administration bypasses the first pass metabolism by the liver, resulting (S)-norketamine concentrations are lower when compared to similar oral or rectal dosing.

While oral (S)-ketamine administration may result in higher concentrations of (S)-norketamine, oral transmucosal administration possesses the additional advantage of a rapid (S)-ketamine concentration and therefore more suitable for the treatment of breakthrough pain. This means high concentration of (S)-ketamine and fast onset of action (short Tmax 2-3 minutes) following the administration of (S)-Ketamine FOT.

High inter-individual variability in peak plasma concentrations of (S)-ketamine (and (S)-norketamine) was observed following the administration of intranasal S-ketamine. This variability may be explained by the obvious problem for some patients to sniff the complete study dose (3 ml). Patients were found to swallowed a substantial portion of (S)-ketamine. The swallowed proportion of the medication subsequently underwent excessive first pass metabolism in the liver, thereby causing increased (S) norketamine concentrations. Therefore the precise amount of intranasally applied (S)-ketamine finally remains unclear, and therefore an unreliable administration regime.

### Oral (S)-Ketamine Using Injection Solution—Summary of Prior Disclosures

An overview of the available clinical data on the use of oral ketamine in chronic pain management reveals that no consistent dose-response relationship was obtained using oral administration of an injection solution.

A recommended starting dosage in ketamine-naïve patients is 0.5 mg/kg racemic ketamine or 0.25 mg/kg S-ketamine as a single oral dose. The dosage is increased by the same amount if required. For a continuous analgesic effect it is usually given 3-4 times daily. The injection fluid was administered orally.

The pharmacologically active metabolite norketamine is believed to contribute to the analgesic effect of oral ketamine.

When used in chronic pain management routes of administration include parenteral (intravenous, subcutaneous, intramuscular, epidural, intra-articular), oral, topical, intranasal and sublingual. No disclosure is apparent of an oral transmucosal administration regime.

Orally administered ketamine undergoes extensive first-pass metabolism in the liver, resulting in a bioavailability of approximately 16%. Oral administration of ketamine is associated with higher serum levels of norketamine compared to other routes of administration. The elimination half-life is 2-3 h for ketamine and approximately 4 h for norketamine. Oral formulations of ketamine are not commercially available. The parenteral formulation is given as an oral solution or an extemporaneous preparation is made.

A literature review (Blonk et al.) reveals that 22 non-comparative Clinical studies or anecdotal reports with a total of 166 patients received oral ketamine in the period 1994 up to 2008. The chronic pain patients had a broad range of pain types. In most cases the pain was diagnosed as neuropathic pain or as having a neuropathic component.

Two approaches to pain treatment with oral ketamine were used: Either the patient started directly with oral ketamine with a low daily dose which, based on clinical effect and/or adverse effects, is increased. Or the patient started with parenteral ketamine, either a single test dose or continuous treatment with usually intravenous or subcutaneous ketamine, after which the patient is switched to an equipotent oral dose of ketamine.

The effective daily dosages ranged from (approximately) 45 mg to 1000 mg. There was no consistent dose-response relation. The number of divided doses necessary for continuous analgesic effect also ranged from once daily up to a frequency of 6 times daily (on average 3-4 times daily). The duration of effect after a single dose (if there was any effect) ranged from a few hours to 24 h or more.

The studies reveal that patients have limited benefits from the use of oral Ketamine. The oral bioavailability of ketamine defined as area under plasma concentration time curve (AUC), after a single oral dose of 0.5 mg/kg is about 20% of the availability after an intravenous injection.

The oral bioavailability of norketamine is similar between the two types of administrations, with much higher peak plasma concentrations (200 ng/ml) reached after oral administration.

When ketamine is administered as a racemic mixture, both S-norketamine and R-norketamine is formed. S-norketamine is approximately five times weaker than S-ketamine with respect to analgesic and anaesthetic effect.

Analgesic effects of ketamine were observed with plasma levels of 100-200 ng/ml (sum of S- and R-isomer) following intramuscular and intravenous administration. Effective analgesia following oral dose occurs at much lower concentrations of ketamine (40 ng/ml). Considering this and the relatively high plasma concentrations of norketamine reached, norketamine is believed to contribute to the analgesic effects of orally administered ketamine.

In a ketamine-naïve patient, oral administration of ketamine can start with a single dose of 0.5 mg/kg ketamine racemic mixture or 0.25 mg/kg S-ketamine to evaluate the effect on pain relief and the duration of effect. Doses can be increased in steps of 0.5 or 0.25 mg/kg according to the efficacy and adverse effects respectively. The average dosing frequency of 3-4 times daily found in clinical studies corresponds with the elimination half-lives of ketamine (2-3) and norketamine (4 h).

### Further Embodiments Regarding Administration and Dosage According to the Present Invention:

The methods and compositions of the instant invention are intended for treating preferably human individuals.

The term “treating” as used herein refers to the lessening or alleviation of symptoms of particular disorder in an individual or the improvement of an ascertainable measurement associated with a particular disorder.

In one aspect, the invention provides pharmaceutical composition comprising an NMDA receptor (N-Methyl-D-Aspartate Receptor) Antagonist and interacting with opioid receptor, adrenergic receptors, muscarinic receptors, as well as voltage-sensitive calcium ion channels and a pharmaceutically acceptable carrier.

The term “pharmaceutically acceptable carrier” refers to a carrier that may be administered to a patient, together with a compound of this invention, and which does not destroy the pharmacological activity of the compound of this invention and is nontoxic when administered in doses sufficient to deliver a therapeutic amount of the compound.

The term “pharmaceutical composition” refers to a combination of the agent as described herein with a pharmaceutically acceptable carrier, preferably suitable for oral transmucosal administration. The phrase “pharmaceutically-acceptable” refers to molecular entities and compositions that do not produce a severe allergic or similar untoward reaction when administered to a human. As used herein, “carrier” includes any and all solvents, dispersion media, vehicles, coatings, diluents, antibacterial and antifungal agents, isotonic and absorption delaying agents, buffers, carrier solutions, suspensions, polymers, colloids, and the like. The use of such media and agents for pharmaceutical active substances is well known in the art. Supplementary active ingredients can also be incorporated into the compositions. A pharmaceutical composition of the present invention can include pharmaceutically acceptable salts of the components therein. The pharmaceutical composition containing the active ingredient may be in a form suitable for topical or transmucosal oral use, for example, as tablets, troches, lozenges, aqueous or oily suspensions, dispersible powders or granules, emulsions, hard or soft capsules, nanocarriers, liposomes, gels, lollipops, mucosal adhesives, or syrups or elixirs. Compositions intended for topical or oral use may be prepared according to any method known to the art for the manufacture of pharmaceutical compositions and such compositions. Tablets may contain the active ingredient in admixture with non-toxic pharmaceutically acceptable excipients which are suitable for the manufacture of tablets.

Pharmaceutically acceptable carriers that may be used in the pharmaceutical compositions of the invention include, but are not limited to, ion exchangers, starches, lactose, cane-sugar, glucose, mannitol and silica, the binder is preferably carboxymethylcellulose, alginate, gelatin, polyvinylpyrrolidone, the humectant is preferably glycerol, the disintegrant is preferably agar, calcium carbonate and sodium carbonate, the dissolution retarder is preferably paraffin, and the absorption enhancer is preferably a quaternary ammonium compound, the wetting agent is preferably cetyl alcohol and glycerol monostearate, the adsorbent is preferably kaolin and bentonite, and the lubricant is preferably talc, calcium and/or magnesium stearate, a solid polyethylene glycol or concerns mixtures of the materials mentioned above.

Pharmaceutically acceptable inorganic salts of S-Ketamine include salts prepared from inorganic acids such as hydrochloric acid, nitric acid, phosphoric acid, sulphuric acid, boric acid, hydrofluoric acid, and hydrobromic acid.

Pharmaceutically acceptable organic salts of S-Ketamine include salts prepared from organic acids such as acetic, trifluoroacetic, propionic, succinic, glycolic, stearic, lactic, malic, tartaric, citric, ascorbic, and amino acid salts such as arginate, asparginate, and glutamate, and combinations comprising one or more of the foregoing salts.

The pharmaceutical compositions of this invention may be administered via oral transmucosal administration routes, orally, parenterally, by inhalation spray, topically, rectally, nasally, buccally, vaginally or via an implanted reservoir. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of sterile injectable preparation, for example, as a composition with a tolerable vehicles and solvents such as mannitol, water, Ringers solution, and isotonic sodium chloride solution. For liquid solutions the compounds or composition may be present together with water, ethyl alcohol, propylene glycol, suspending agents, e.g. ethoxylated isostearyl alcohols, polyoxyethylene sorbitol and sorbitan esters, microcrystalline cellulose, aluminum metahydroxide, bentonite, agar, tragacanth, or mixtures of these substances.

The pharmaceutical compositions may be orally administered in any orally acceptable dosage form including but not limited to, oral transmucosal carriers, Buccal delivery compositions, sublingual formulations, Orodispersible Tablets (ODT), Orodispersible Films (ODF), Orodispersible Granules (Micro-Pellets), Fast Oral Transmucosal (FOT), capsules, tablets, an aqueous suspensions and solutions.

The pharmaceutical compositions may also be administered in the form of suppositories for rectal administration.

Topical or transmucosal administration of the pharmaceutical compositions of this invention is especially useful when the desired treatment involves areas or organs readily accessible by topical or transmucosal application; carriers for topical or transmucosal administration of the compounds of this invention include, but are not limited to a humectant (such as for example propylene glycol, glycerin, butylen glycol or polyethylene glycol), a buffer (such as for example citric acid aqueous solution, ammonium hydroxide solution phosphate buffer, borate buffer or carbonate buffer), a lubricant (such as for example cyclomethicone, dimethycone, castor oil, Iso propyl miristate, caprylic/capric triglyceride or octyl octanoate), an emulsifier (such as for example cetyl alcohol, glyceryl stearate, PEG-75 stearate, Ceteth-20, Steareth-20, Bis-PEG/PPG-16/16 PEG/PPG-16/16 dimethicone, sorbitan mono-oleate or alkyl poly glucoside), a moisturize (such as for example sodium PCA, sodium hyaluronate, panthenol or sodium latate), a soothing agent (such as for example natural herbal extracts such as Anthemis Nobilis flower extract, Chamomilla Recutita, Hamamelis Virginiana, burdock root, Argireline, Arnica Montana Extract, Shea Butter or aloe vera), a perfume, an exfoliating agent (such as for example polyethylene, glycolic acid 70%), a filler, an anti-irritating agent (such as for example allantoin), a chelating agent (such as for example EDTA), a preservative (such as for example imidazolidinyl urea, potassium sorbate, phenoxyethanol, methyl paraben, propyl paraben or benzyl alcohol), a detergent (such as for example polysorbate 20, sodium dodecyl sulfate or ceterimonium chlorid), a coloring agent, an antimicrobial agent (such as for example SD alcohol 40 or Chlorhexidine gluconate), a thickening agent (such as for example xanthan gum, guar gum, carboxy methyl cellulose, Carbomer or ethyl cellulose) and any combinations thereof.

The pharmaceutical compositions may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solublilizing or dispersing agents known in the art.

The pharmaceutical compositions of the present invention may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a therapeutic effect. Generally, out of one hundred percent, this amount will range in some embodiments from about 1 percent to about ninety-nine percent of active ingredient, in some embodiments from about 5 percent to about 70 percent, and in some embodiments from about 10 percent to about 30 percent.

The selected dosage level will depend upon a variety of factors including the activity of the particular compound of the present invention employed, or the ester, salt or amide thereof, the route of administration, the time of administration, the rate of excretion of the particular compound being employed, the duration of treatment, other drugs, compounds and/or materials used in combination with the particular compound employed, the age, gender, weight, condition, general health and prior medical history of the patient being treated, and like factors well known in the medical art.

In general, a suitable daily dose of a compound of the invention will be that amount of the compound that is the lowest dose effective to produce a therapeutic effect. Such an effective dose will be generally depend upon the factors described above. If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In some embodiments, the active compound will be administered once daily.

In another aspect of the invention, the compounds of the invention are administered alone or co-administered with another therapeutic agent. As used herein, the phrase “co-administration” refers to any form of administration of two or more different therapeutic compounds such that the desired effect is obtained. The different therapeutic compounds may be administered either in the same formulation or in separate formulation, either concomitantly or sequentially. Thus, an individual who receives such treatment may benefit from a combined effect of different therapeutic compounds. Co-administration includes simultaneous or sequential administration of two or more compounds which may have synergistic, additive and/or different therapeutic effects.

### FIGURES

The figures provided herein represent examples of particular embodiments of the invention and are not intended to limit the scope of the invention. The following drawings form part of the present specifications and are included to further illustrate aspects of the present invention. The invention may be better understood by reference to the drawings in combination with the detailed description of the specific embodiments presented herein.

### BRIEF DESCRIPTION OF THE DRAWINGS

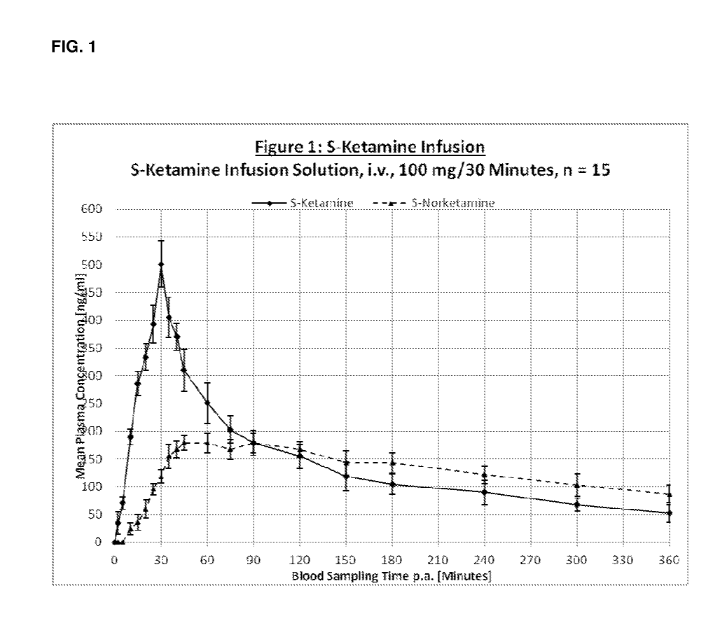

FIG. 1. Mean S-ketamine and nor S-ketamine plasma concentrations (+/−SD) following single dose administration of S-ketamine infusion, 100 mg/30 Minutes to 15 healthy volunteers.

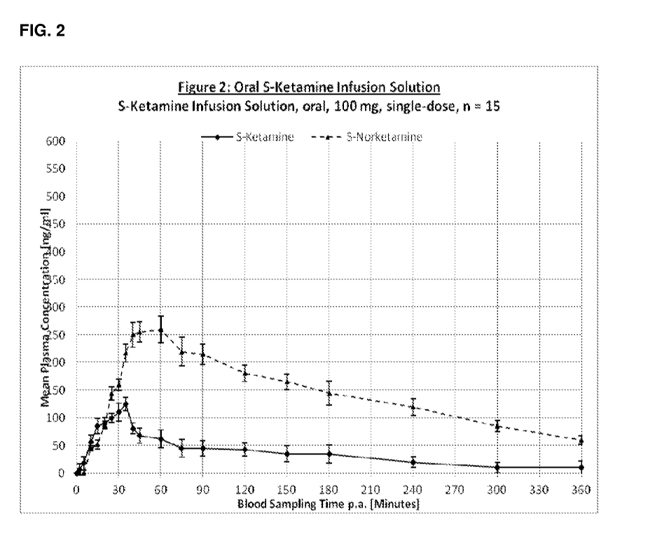

FIG. 2. Mean S-ketamine and nor S-ketamine plasma concentrations (+/−SD) following single dose oral administration of 100 mg S-ketamine infusion solution, to 15 healthy volunteers.

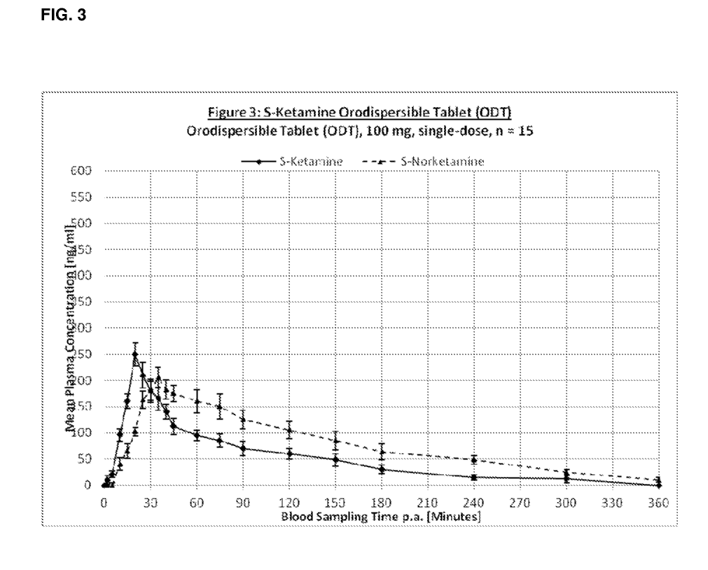

FIG. 3. Mean S-ketamine and nor S-ketamine plasma concentrations (+/−SD) following single dose oral administration of 100 mg S-ketamine orodispersible Tablet (ODT), to 15 healthy volunteers.

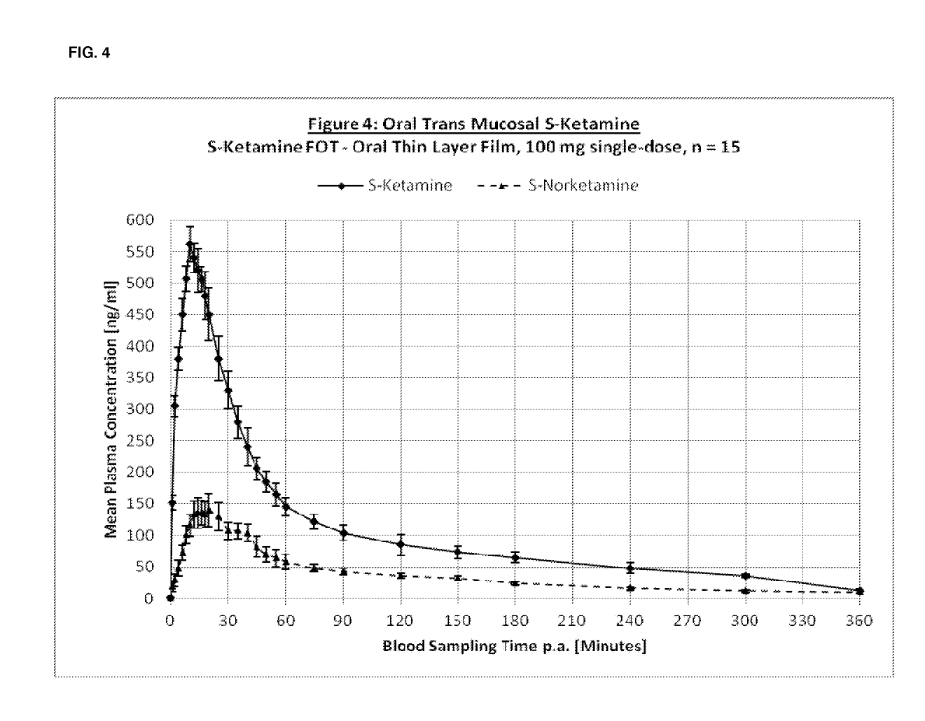

FIG. 4. Mean S-ketamine and nor S-ketamine plasma concentrations (+/−SD) following single dose oral administration of 100 mg S-ketamine Thin Layer Film, to 15 healthy volunteers.

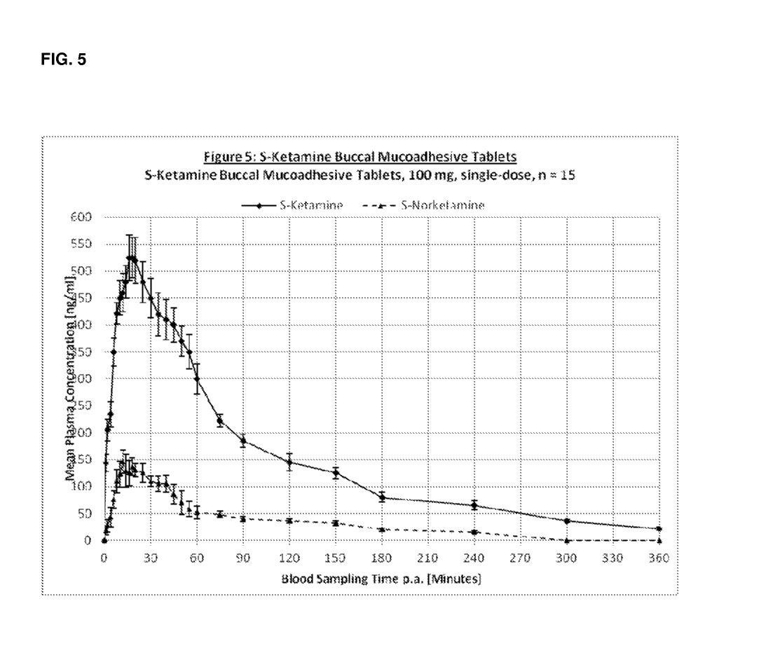

FIG. 5. Mean S-ketamine and nor S-ketamine plasma concentrations (+/−SD) following single dose oral administration of 100 mg S-ketamine Buccal Mucoadhesive Tablets, to 15 healthy volunteers.

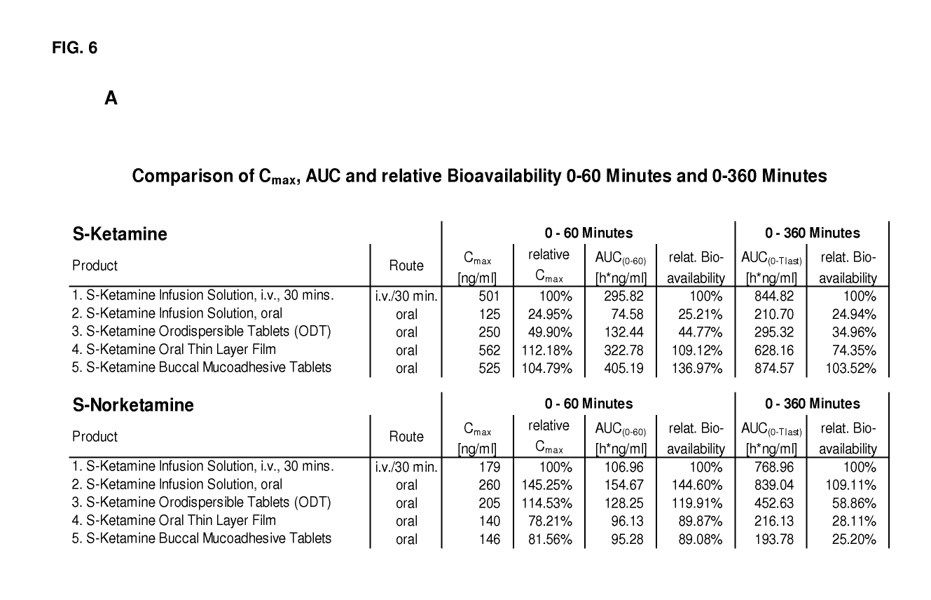

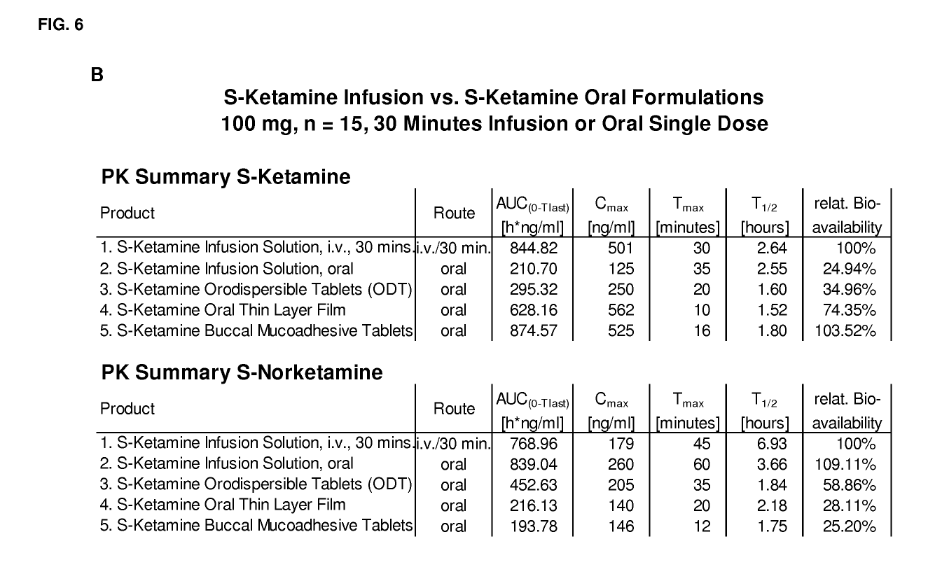

FIG. 6. Comparison of Cmax, AUC and relative bioavailability at 0-60 minutes and 0-360 minutes for each of the administration methods shown in Example 1.

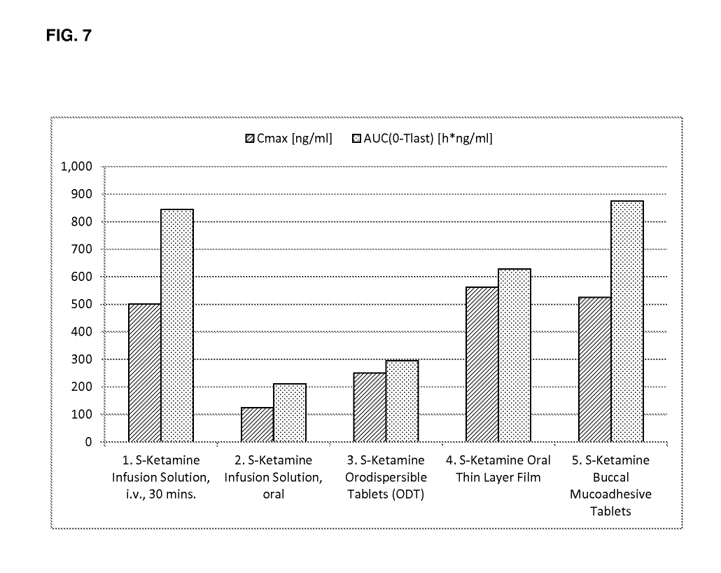

FIG. 7. PK Summary S-Ketamine Infusion vs. S-Ketamine Oral Formulations; S-Ketamine Cₘₐₓ and AUC.

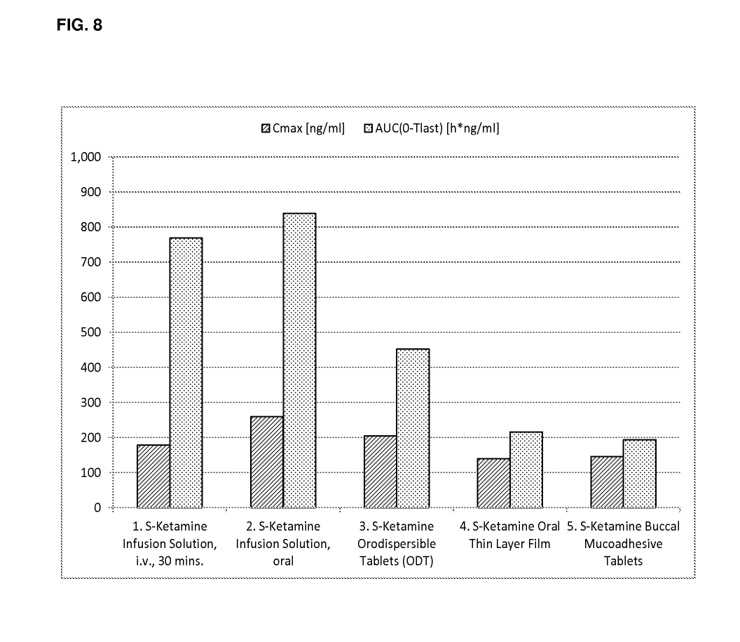

FIG. 8. S-Norketamine Cₘₐₓ and AUC.

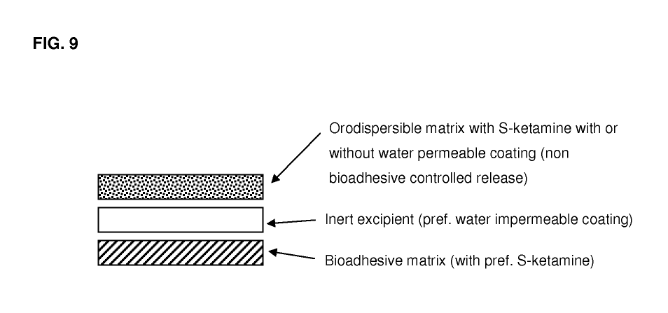

FIG. 9. Schematic representation of the FOT matrix designed for the combined Mucoadhesive system (mucoadhesive patch/tablet) and orodispersible matrix with S-ketamine (bidirectional release of the active agent S-ketamine to the buccal mucosa and the cavity mucosa).

### EXAMPLES

The examples provided herein represent practical support for particular embodiments of the invention and are not intended to limit the scope of the invention. The examples are to be considered as providing a further description of potentially preferred embodiments that demonstrate the relevant technical working of one or more non-limiting embodiments.

### Example 1

### Pharmacokinetics (PK) of S-Ketamine Pharmaceutical Formulations

The following pharmaceutical formulations of S-Ketamine were tested:
1. S-Ketamine Infusion Solution i.v.
2. S-Ketamine Infusion Solution oral
3. S-Ketamine Orodispersible Tablet (ODT)
4. S-Ketamine Thin Layer Film
5. S-Ketamine Buccal Mucoadhesive Tablets

### Study Design:

Open randomized, 5-way cross-over study in 15 healthy volunteers to assess and compare the Pharmacokinetics of S-Ketamine Infusion Solution i.v., S-Ketamine Infusion Solution oral, S-Ketamine Orodispersible Tablets (ODT), S-Ketamine Thin Layer Film, and S-Ketamine Buccal Mucoadhesive Tablets.

Treatment group 1: (the active reference drug): INTRAVENOUS 100 mg S-ketamine given by infusion pump over 30 minutes. PK measurements for 6-h.

Treatment group 2: ORAL 100 mg S-ketamine injection solution with lemonade (total volume 100 ml), PK measurements for 6-h.

Treatment group 3: ORAL TRANSMUCOSAL 100 mg S-ketamine Orodispersible Tablets (ODT), PK measurements for 6-h.

Treatment group 4: ORAL TRANSMUCOSAL 100 mg S-ketamine Thin Layer Film, PK measurements for 6-h.

Treatment group 5: ORAL TRANSMUCOSAL—100 mg S-ketamine Buccal Mucoadhesive Tablets, PK measurements for 6-h.

### Measurements on the Treatment Day:

Blood sampling/determination of S-ketamine and S-norketamine in plasma samples was carried out. Venous blood samples for measurement of plasma concentrations of S-ketamine and its active metabolite S-norketamine were obtained at the following sampling times:

t=0 (baseline): just prior to start of infusion or drug administration, respectively.

Treatment group 1: t=0 (pre-dose), 2, 5, 10, 15, 20, 25, 30 (end of infusion), 35, 40, 45, 60, 75, 90, 120, 150, 180, 240, 300 and 360 minutes after start of infusion.

Treatment group 2: t=0 (pre-dose), 2, 5, 10, 15, 20, 25, 30, 35, 40, 45, 60, 75, 90, 120, 150, 180, 240, 300 and 360 minutes p.a.

Treatment group 3: t=0 (pre-dose), 2, 5, 10, 15, 20, 25, 30, 35, 40, 45, 60, 75, 90, 120, 150, 180, 240, 300 and 360 minutes p.a.

Treatment group 4: t=0 (pre-dose), 1, 2, 4, 6, 8, 10, 12, 14, 16, 18, 20, 25, 30, 35, 40, 45, 50, 55, 60, 75, 90, 120, 150, 180, 240, 300 and 360 minutes p.a.

Treatment group 5: t=0 (pre-dose), 1, 2, 4, 6, 8, 10, 12, 14, 16, 18, 20, 25, 30, 35, 40, 45, 50, 55, 60, 75, 90, 120, 150, 180, 240, 300 and 360 minutes p.a.

Plasma was separated after 15 min of blood collection and stored at −20 C° until analysis. Analysis was performed using validated chiral high performance liquid chromatography (HPLC) method. The lower limit of quantitation (LLQ) was 10 ng S-ketamine/ml plasma and 10 ng S-norketamine/ml plasma. The lower limit of detection was 3 ng/ml plasma for S-ketamine and S-norketamine.

Results from Example 1 (FIGS. 1-5):

### Treatment Group 1:

TABLE 1
refer FIG. 1-Data:
S-Ketamine Infusion Solution, i.v. Infusion, 100 mg/30 Minutes, n = 15

| | Blood Sampling | | | | | |
|---|---|---|---|---|---|---|
| | S-Ketamine | | | S-Norketamine | | |
| | Plasma | | | Plasma | | |
| Time | Concentration | | AUC_{(T1-T2)} | Concentration | | AUC_{(T1-T2)} |
| [Minutes] | [ng/ml] | ±SD | [h * ng/ml] | [ng/ml] | ±SD | [h * ng/ml] |
| 0 | 0 | 0 | 0 | 0 | 0 | 0 |
| 2 | 35 | 20 | 0.58 | 0 | 0 | 0.00 |
| 5 | 71 | 11 | 2.65 | 0 | 0 | 0.00 |
| 10 | 190 | 14 | 10.88 | 24 | 11 | 1.00 |
| 15 | 286 | 22 | 19.83 | 36 | 14 | 2.50 |
| 20 | 334 | 24 | 25.83 | 60 | 16 | 4.00 |
| 25 | 393 | 34 | 30.29 | 96 | 10 | 6.50 |
| 30 | 501 | 42 | 37.25 | 119 | 12 | 8.96 |
| 35 | 405 | 36 | 37.75 | 155 | 22 | 11.42 |
| 40 | 370 | 24 | 32.29 | 167 | 15 | 13.42 |
| 45 | 310 | 38 | 28.33 | 179 | 13 | 14.42 |
| 60 | 251 | 36 | 70.13 | 179 | 18 | 44.75 |
| 75 | 203 | 24 | 56.75 | 167 | 18 | 43.25 |
| 90 | 179 | 18 | 47.75 | 179 | 22 | 43.25 |
| 120 | 155 | 22 | 83.50 | 167 | 14 | 86.50 |
| 150 | 119 | 26 | 68.50 | 143 | 22 | 77.50 |
| 180 | 105 | 18 | 56.00 | 143 | 18 | 71.50 |
| 240 | 90 | 22 | 97.50 | 122 | 16 | 132.50 |
| 300 | 68 | 12 | 79.00 | 103 | 20 | 112.50 |
| 360 | 52 | 16 | 60.00 | 87 | 16 | 95.00 |
| | AUC_{(0-Tlast)} | 844.82 | [h * ng/ml] | AUC_{(0-Tlast)} | 768.96 | [h * ng/ml] |
| | Cₘₐₓ | 501 | [ng/ml] | Cₘₐₓ | 179 | [ng/ml] |
| | Tₘₐₓ | 30 | [Minutes] | Tₘₐₓ | 45 | [Minutes] |
| | T_{1/2} | 2.64 | [Hours] | T_{1/2} | 6.93 | [Hours] |

### Treatment Group 2:

TABLE 2
refer FIG. 2-Data:
Oral S-Ketamine Infusion Solution, 100 mg, single-dose, n = 15

| | Blood Sampling | | | | | |
|---|---|---|---|---|---|---|
| | S-Ketamine | | | S-Norketamine | | |
| | Plasma | | | Plasma | | |
| Time | Concentration | | AUC_{(T1-T2)} | Concentration | | AUC_{(T1-T2)} |
| [Minutes] | [ng/ml] | ±SD | [h * ng/ml] | [ng/ml] | ±SD | [h * ng/ml] |
| 0 | 0 | 0 | 0 | 0 | 0 | 0 |
| 2 | 7 | 10 | 0.12 | <LLQ | 0 | 0.00 |
| 5 | 18 | 12 | 0.63 | <LLQ | 0 | 0.00 |
| 10 | 57 | 12 | 3.13 | 45 | 5 | 1.88 |
| 15 | 85 | 14 | 5.92 | 52 | 8 | 4.04 |
| 20 | 90 | 10 | 7.29 | 88 | 6 | 5.83 |
| 25 | 100 | 8 | 7.92 | 144 | 12 | 9.67 |
| 30 | 110 | 16 | 8.75 | 160 | 10 | 12.67 |
| 35 | 125 | 12 | 9.79 | 217 | 16 | 15.71 |
| 40 | 81 | 10 | 8.58 | 250 | 22 | 19.46 |
| 45 | 68 | 14 | 6.21 | 255 | 18 | 21.04 |
| 60 | 62 | 16 | 16.25 | 260 | 24 | 64.38 |
| 75 | 45 | 16 | 13.38 | 220 | 26 | 60.00 |
| 90 | 45 | 14 | 11.25 | 215 | 18 | 54.38 |
| 120 | 43 | 12 | 22.00 | 180 | 15 | 98.75 |
| 150 | 35 | 14 | 19.50 | 165 | 14 | 86.25 |
| 180 | 35 | 16 | 17.50 | 145 | 22 | 77.50 |
| 240 | 20 | 10 | 27.50 | 120 | 15 | 132.50 |
| 300 | 10 | 10 | 15.00 | 85 | 10 | 102.50 |
| 360 | 10 | 12 | 10.00 | 60 | 8 | 72.50 |
| | AUC_{(0-Tlast)} | 210.70 | [h * ng/ml] | AUC_{(0-Tlast)} | 839.04 | [h * ng/ml] |
| | Cₘₐₓ | 125 | [ng/ml] | Cₘₐₓ | 260 | [ng/ml] |
| | Tₘₐₓ | 35 | [Minutes] | Tₘₐₓ | 60 | [Minutes] |
| | T_{1/2} | 2.55 | [Hours] | T_{1/2} | 3.66 | [Hours] |

### Treatment Group 3:

TABLE 3
refer FIG. 3-Data:
S-Ketamine Orodispersible Tablets (ODT), 100 mg, single-dose, n = 15

| | Blood Sampling | | | | | |
|---|---|---|---|---|---|---|
| | S-Ketamine | | | S-Norketamine | | |
| | Plasma | | | Plasma | | |
| Time | Concentration | | AUC_{(T1-T2)} | Concentration | | AUC_{(T1-T2)} |
| [Minutes] | [ng/ml] | ±SD | [h * ng/ml] | [ng/ml] | ±SD | [h * ng/ml] |
| 0 | 0 | 0 | 0 | 0 | 0 | 0 |
| 2 | 10 | 8 | 0.17 | 0 | 0 | 0.00 |
| 5 | 21 | 6 | 0.78 | 15 | 5 | 0.38 |
| 10 | 96 | 12 | 4.88 | 40 | 12 | 2.29 |
| 15 | 160 | 14 | 10.67 | 65 | 14 | 4.38 |
| 20 | 250 | 22 | 17.08 | 102 | 8 | 6.96 |
| 25 | 210 | 24 | 19.17 | 163 | 16 | 11.04 |
| 30 | 180 | 18 | 16.25 | 180 | 22 | 14.29 |
| 35 | 165 | 22 | 14.38 | 205 | 20 | 16.04 |
| 40 | 140 | 14 | 12.71 | 182 | 18 | 16.13 |
| 45 | 112 | 16 | 10.50 | 175 | 16 | 14.88 |
| 60 | 95 | 10 | 25.88 | 160 | 22 | 41.88 |
| 75 | 85 | 12 | 22.50 | 150 | 24 | 38.75 |
| 90 | 70 | 14 | 19.38 | 125 | 18 | 34.38 |
| 120 | 60 | 10 | 32.50 | 105 | 16 | 57.50 |
| 150 | 48 | 12 | 27.00 | 85 | 18 | 47.50 |
| 180 | 30 | 8 | 19.50 | 64 | 15 | 37.25 |
| 240 | 15 | 5 | 22.50 | 48 | 8 | 56.00 |
| 300 | 12 | 8 | 13.50 | 24 | 6 | 36.00 |
| 360 | <LLQ | 0 | 6.00 | 10 | 4 | 17.00 |
| | AUC_{(0-Tlast)} | 295.32 | [h * ng/ml] | AUC_{(0-Tlast)} | 452.63 | [h * ng/ml] |
| | Cₘₐₓ | 250 | [ng/ml] | Cₘₐₓ | 205 | [ng/ml] |
| | Tₘₐₓ | 20 | [Minutes] | Tₘₐₓ | 35 | [Minutes] |
| | T_{1/2} | 1.60 | [Hours] | T_{1/2} | 1.84 | [Hours] |

### S-Ketamine Orodispersible Tablets (ODT)

### S-Ketamine Results:

1. It is surprising and unexpected that the relative bioavailability for S-ketamine following the administration of 100 mg S-ketamine Orodispersible Tablets according to the present invention was very high and approximately % 177,58(132.44:74.58 h·ng/ml—60 minutes) and 140.16 (295.32:210.70 h·ng/ml—360 minutes) in comparison to the oral S-ketamine infusion solution.
2. It is surprising and unexpected that the maximal concentration Cmaxof S-ketamine in plasma following the administration of 100 mg S-ketamine Orodispersible Tablets according to the present invention was very high and approximately 200.0% (250:125 ng/ml) in comparison to the oral intravenous S-ketamine infusion solution.

### Treatment Group 4:

TABLE 4
refer FIG. 4-Data:
S-Ketamine Oral Thin Layer Film, 100 mg single-dose, n = 15

| | Blood Sampling | | | | | |
|---|---|---|---|---|---|---|
| | S-Ketamine | | | S-Norketamine | | |
| | Plasma | | | Plasma | | |
| Time | Concentration | | AUC_{(T1-T2)} | Concentration | | AUC_{(T1-T2)} |
| [Minutes] | [ng/ml] | ±SD | [h * ng/ml] | [ng/ml] | ±SD | [h * ng/ml] |
| 0 | 0 | 0 | 0 | 0 | 0 | 0 |
| 1 | 152 | 12 | 1.27 | 19 | 8 | 0.16 |
| 2 | 305 | 16 | 3.81 | 29 | 10 | 0.40 |
| 4 | 380 | 18 | 11.42 | 48 | 12 | 1.28 |
| 6 | 450 | 26 | 13.83 | 72 | 12 | 2.00 |
| 8 | 507 | 20 | 15.95 | 101 | 14 | 2.88 |
| 10 | 562 | 28 | 17.82 | 116 | 18 | 3.62 |
| 12 | 540 | 24 | 18.37 | 133 | 22 | 4.15 |
| 14 | 520 | 35 | 17.67 | 136 | 24 | 4.48 |
| 16 | 506 | 20 | 17.10 | 136 | 20 | 4.53 |
| 18 | 480 | 38 | 16.43 | 133 | 20 | 4.48 |
| 20 | 450 | 42 | 15.50 | 140 | 26 | 4.55 |
| 25 | 380 | 36 | 34.58 | 130 | 22 | 11.25 |
| 30 | 330 | 30 | 29.58 | 107 | 14 | 9.88 |
| 35 | 280 | 26 | 25.42 | 107 | 12 | 8.92 |
| 40 | 240 | 30 | 21.67 | 104 | 14 | 8.79 |
| 45 | 206 | 18 | 18.58 | 82 | 16 | 7.75 |
| 50 | 185 | 16 | 16.29 | 70 | 12 | 6.33 |
| 55 | 165 | 18 | 14.58 | 64 | 14 | 5.58 |
| 60 | 145 | 14 | 12.92 | 58 | 12 | 5.08 |
| 75 | 122 | 12 | 33.38 | 48 | 6 | 13.25 |
| 90 | 104 | 12 | 28.25 | 42 | 5 | 11.25 |
| 120 | 85 | 16 | 47.25 | 36 | 4 | 19.50 |
| 150 | 73 | 10 | 39.50 | 32 | 4 | 17.00 |
| 180 | 65 | 9 | 34.50 | 24 | 3 | 14.00 |
| 240 | 48 | 8 | 56.50 | 16 | 2 | 20.00 |
| 300 | 36 | 3 | 42.00 | 12 | 2 | 14.00 |
| 360 | 12 | 3 | 24.00 | 10 | 2 | 11.00 |
| | AUC_{(0-Tlast)} | 628.16 | [h * ng/ml] | AUC_{(0-Tlast)} | 216.13 | [h * ng/ml] |
| | Cₘₐₓ | 562 | [ng/ml] | Cₘₐₓ | 140 | [ng/ml] |
| | Tₘₐₓ | 10 | [Minutes] | Tₘₐₓ | 20 | [Minutes] |
| | T_{1/2} | 1.52 | [Hours] | T_{1/2} | 2.18 | [Hours] |

### S-Ketamine Thin Layer Film

### S-Ketamine Results:

1. It is surprising and unexpected that the relative bioavailability for S-ketamine following the administration of 100 mg transbuccal S-ketamine Thin Layer Formulation according to the present invention was very high and approximately 109% (60 minutes), and 74.35% (360 minutes) in comparison to the intravenous S-ketamine infusion for 30 minutes.
2. It is surprising and unexpected that the relative maximal concentration of S-ketamine in plasma following the administration of 100 mg transbuccal S-ketamine Thin Layer Formulation according to the present invention was very high and approximately 112.18% in comparison to the intravenous S-ketamine infusion for 30 minutes.
3. It is surprising and unexpected that the time to maximal concentration Tmax of S-ketamine following the administration of 100 mg transbuccal S-ketamine Thin Layer formulation according to the present invention was shorter than of the intravenous S-ketamine infusion for 30 minutes (33.33%).
4. It is surprising and unexpected that the relative bioavailability for S-ketamine following the administration of 100 mg transbuccal S-ketamine Thin Layer Formulation according to the present invention was very high and approximately 432.80% (322.78:74.58 h·ng/ml) in comparison to the oral S-ketamine infusion solution.
5. It is surprising and unexpected that the maximal concentration Cmax of S-ketamine in plasma following the administration of 100 mg transbuccal S-ketamine Thin Layer Formulation according to the present invention was very high and approximately 449.6% (562:125 ng/ml) in comparison to the oral intravenous S-ketamine infusion solution.

### S-Norketamine Results:

1. It is surprising and unexpected that the relative bioavailability of S-norketamine following the administration of 100 mg transbuccal S-ketamine Thin Layer Formulation according to the present invention was very low 28.11% (360 minutes) in comparison to the intravenous S-ketamine infusion for 30 minutes.
2. It is surprising and unexpected that the relative maximal concentration of S-norketamine in plasma following the administration of 100 mg transbuccal S-ketamine Thin Layer Formulation according to the present invention was very low and approximately 78.21% in comparison to the intravenous S-ketamine infusion for 30 minutes.
3. It is surprising and unexpected that the time to maximal concentration Tmax of S-norketamine following the administration of 100 mg transbuccal S-ketamine Thin Layer formulation according to the present invention was shorter than of the intravenous S-ketamine infusion for 30 minutes 44.4% (20:45 minutes).
4. It is surprising and unexpected that the relative bioavailability for S-norketamine following the administration of 100 mg transbuccal S-ketamine Thin Layer Formulation according to the present invention was very low and approximately 62.15% (96.13:154.67 h·ng/ml—60 minutes and 25.76% (216.13:839.04—360 minutes) in comparison to the oral S-ketamine infusion solution.
5. It is surprising and unexpected that the maximal concentration Cmaxof S-norketamine in plasma following the administration of 100 mg transbuccal S-ketamine Thin Layer Formulation according to the present invention was very low and approximately 53.85% (140:260 ng/ml) in comparison to the oral intravenous S-ketamine infusion solution.

### Treatment Group 5:

TABLE 5
refer FIG. 5-Data:
S-Ketamine Buccal Mucoadhesive Tablets 100 mg, single-dose, n = 15

| | Blood Sampling | | | | | |
|---|---|---|---|---|---|---|
| | S-Ketamine | | | S-Norketamine | | |
| | Plasma | | | Plasama | | |
| Time | Concentration | | AUC_{(T1-T2)} | Concentration | | AUC_{(T1-T2)} |
| [Minutes] | [ng/ml] | ±SD | [h * ng/ml] | [ng/ml] | ±SD | [h * ng/ml] |
| 0 | 0 | 0 | 0 | 0 | 0 | 0 |
| 1 | 144 | 16 | 1.20 | 18 | 8 | 0.15 |
| 2 | 205 | 20 | 2.91 | 27 | 12 | 0.38 |
| 4 | 234 | 24 | 7.32 | 43 | 18 | 1.17 |
| 6 | 350 | 26 | 9.73 | 76 | 16 | 1.98 |
| 8 | 421 | 20 | 12.85 | 110 | 22 | 3.10 |
| 10 | 450 | 32 | 14.52 | 123 | 24 | 3.88 |
| 12 | 460 | 36 | 15.17 | 146 | 22 | 4.48 |
| 14 | 480 | 30 | 15.67 | 129 | 30 | 4.58 |
| 16 | 525 | 42 | 16.75 | 125 | 24 | 4.23 |
| 18 | 525 | 38 | 17.50 | 137 | 16 | 4.37 |
| 20 | 520 | 42 | 17.42 | 130 | 12 | 4.45 |
| 25 | 480 | 38 | 41.67 | 125 | 18 | 10.63 |
| 30 | 450 | 36 | 38.75 | 110 | 10 | 9.79 |
| 35 | 420 | 40 | 36.25 | 105 | 14 | 8.96 |
| 40 | 410 | 38 | 34.58 | 105 | 16 | 8.75 |
| 45 | 400 | 32 | 33.75 | 86 | 18 | 7.96 |
| 50 | 370 | 28 | 32.08 | 70 | 22 | 6.50 |
| 55 | 350 | 32 | 30.00 | 58 | 14 | 5.33 |
| 60 | 300 | 28 | 27.08 | 52 | 12 | 4.58 |
| 75 | 222 | 12 | 65.25 | 48 | 6 | 12.50 |
| 90 | 185 | 12 | 50.88 | 40 | 5 | 11.00 |
| 120 | 145 | 16 | 82.50 | 36 | 4 | 19.00 |
| 150 | 125 | 10 | 67.50 | 32 | 4 | 17.00 |
| 180 | 80 | 9 | 51.25 | 20 | 3 | 13.00 |
| 240 | 65 | 8 | 72.50 | 16 | 2 | 18.00 |
| 300 | 36 | 3 | 50.50 | <LLQ | 0 | 8.00 |
| 360 | 22 | 3 | 29.00 | <LLQ | 0 | 0.00 |
| | AUC_{(0-Tlast)} | 874.57 | [h * ng/ml] | AUC_{(0-Tlast)} | 193.78 | [h * ng/ml] |
| | Cₘₐₓ | 525 | [ng/ml] | Cₘₐₓ | 146 | [ng/ml] |
| | Tₘₐₓ | 16 | [Minutes] | Tₘₐₓ | 12 | [Minutes] |
| | T_{1/2} | 1.80 | [Hours] | T_{1/2} | 1.75 | [Hours] |

### S-Ketamine Buccal Mucoadhesive Tablets

### S-Ketamine Results:

1. It is surprising and unexpected that the relative bioavailability for S-ketamine following the administration of 100 mg S-ketamine Buccal Mucoadhesive Tablets according to the present invention was very high and approximately 104.9% (60 minutes), and 103.52% (360 minutes) in comparison to the intravenous S-ketamine infusion for 30 minutes.
2. It is surprising and unexpected that the relative maximal concentration of S-ketamine in plasma following the administration of 100 mg S-ketamine Buccal Mucoadhesive Tablets according to the present invention was very high and approximately 104.79% (60 minutes) and 103.52 (360 minutes) in comparison to the intravenous S-ketamine infusion for 30 minutes.
3. It is surprising and unexpected that the time to maximal concentration Tmax of S-ketamine following the administration of 100 mg S-ketamine Buccal Mucoadhesive Tablets according to the present invention was shorter than of the intravenous S-ketamine infusion for 30 minutes (53.33%—16:30 minutes).

4. It is surprising and unexpected that the relative bioavailability for S-ketamine following the administration of 100 mg S-ketamine Buccal Mucoadhesive Tablets according to the present invention was very high and approximately 543.30% (405.19:74.58 h·ng/ml—60 minutes) and 415.1% (874.57:210.70 h·ng/ml—360 minutes) in comparison to the oral S-ketamine infusion solution.

5. It is surprising and unexpected that the maximal concentration Cmaxof S-ketamine in plasma following the administration of 100 mg S-ketamine Buccal Mucoadhesive Tablets according to the present invention was very high and approximately 420.0% (525:125 ng/ml) in comparison to the oral intravenous S-ketamine infusion solution.

### S-Norketamine Results:

1. It is surprising and unexpected that the relative bioavailability of S-norketamine following the administration of 100 mg S-ketamine Buccal Mucoadhesive Tablets according to the present invention was very low 25.20%% (360 minutes) in comparison to the intravenous S-ketamine infusion for 30 minutes.
2. It is surprising and unexpected that the relative maximal concentration of S-norketamine in plasma following the administration of 100 mg S-ketamine Buccal Mucoadhesive Tablets according to the present invention was low and approximately 81.56% in comparison to the intravenous S-ketamine infusion for 30 minutes.
3. It is surprising and unexpected that the time to maximal concentration Tmax of S-norketamine following the administration of 100 mg S-ketamine Buccal Mucoadhesive Tablets according to the present invention was shorter than of the intravenous S-ketamine infusion for 30 minutes 26.7% (12:45 minutes).
4. It is surprising and unexpected that the relative bioavailability for S-norketamine following the administration of 100 mg S-ketamine Buccal Mucoadhesive Tablets according to the present invention was very low and approximately 23.1% (193.78:839.04 h·ng/ml—360 minutes) in comparison to the oral S-ketamine infusion solution.
5. It is surprising and unexpected that the maximal concentration Cmaxof S-norketamine in plasma following the administration of 100 mg S-ketamine Buccal Mucoadhesive Tablets according to the present invention was very low and approximately 56.15% (146:260 ng/ml) in comparison to the oral intravenous S-ketamine infusion solution.

For an additional representation of the PK data we refer to FIG. 6, representing a comparison of Cmax, AUC and relative bioavailability at 0-60 minutes and 0-360 minutes, and to FIGS. 7 and 8, demonstrating Cmaxand AUC values for S-ketamine (FIG. 7) and S-Norketamine (FIG. 8), for each of the administration methods shown in Example 1.

### Example 2

### CRPS-1 Pain—Treatment with S-Ketamine Thin Layer Film-100 Mg

One patient (male, age 67 years) and eligible for this clinical case was diagnosed with CRPS-1 in both arms, as based on the international Association for the study of Pain CRPS-1 criteria. The patient reported pain scores of 5 or higher (on a numerical rating scale (NRS) from 0 to 10, where 0=no pain and 10=worst pain). Exclusion criteria included age<18 years, inability to give informed consent, serious medical disease (e.g., cardiovascular, renal, or liver disease), use of strong opioids or baclofen, pregnancy/lactation, and history of psychosis. The patient was asked not to change his pain medication from the start of the clinical case study until completion of follow up.

### Treatment:

S-ketamine Thin Layer Film, 100 mg S-ketamine was administered twice for one day at 8:00 h and 20:00 pm).

### Measurements:

The primary outcome measure of the study was pain relief as measured by the 11-point NRS ranging from 0 (no pain) to 10 (worst pain), measured 4 times daily (8:00 h, 12:00 h, 16:00 h, and 20:00 h) during treatment and follow-up.

Secondary outcome parameters were psychotropic side effects, nausea, and headache, all scored on a range from 0 (not present) to 10 (unbearable).

### Results:

S-Ketamine produced a reduction in CRPS pain score from 8 to 0 on treatment day 1. On day 2 the patient was pain free. Pain relief lasted until day 4.

### Day 1:

08:00 h:Baseline NRS=8, drug administration

12:00 h:NRS=2, reduction of pain intensity by 6 points

16:00 h:NRS=0, no pain, reduction of pain by 2 points

20:00 h:NRS=0, no pain, drug administration

### Day 2:

08:00 h:NRS=0, no pain

12:00 h:NRS=0, no pain

16:00 h:NRS=0, no pain

20:00 h:NRS=0, no pain

### Day 3:

08:00 h:NRS=0, no pain

12:00 h:NRS=0, no pain

16:00 h:NRS=0, no pain

20:00 h:NRS=0, no pain

### Day 4:

08:00 h:NRS=0, no pain

12:00 h:NRS=0, no pain

16:00 h:NRS=0, no pain

20:00 h:NRS=2, no pain, slight increase of pain by 2 points

It is surprising and unexpected that the reduction of the CRPS pain occurred at the time course according to the pharmacokinetic profile of S-ketamine and S-norketamine.

It is surprising and unexpected that the side effect profile was minimized because the active ingredient S-ketamine was not metabolized immediately in the liver upon the transmucosal absorption. This will have a positive effect on the side effect profile following the repeated administration of high doses of S-ketamine.

### Example 3

### Spontaneous Fibromyalgia Pain—Treatment with S-Ketamine Buccal Mucoadhesive Tablet (100 mg)

One patient (female, age 66) eligible for this clinical case was diagnosed with Fibromyalgia syndrome as based on the 1990 “American College of Rheumatology” criteria: presence of widespread pain and tenderness in at least 11 of 18 tender points on specific muscle-tendon sites, age 18-75 years, spontaneous pain score of 5 or greater, and who had pain scores of 5 or higher based on a numerical rating scale (NRS) from 0 to 10, where 0=no pain and 10=worst pain).

Exclusion criteria included age<18 years, inability to give informed consent, serious medical disease (e.g., cardiovascular, renal, or liver disease), use of strong opioids or baclofen, pregnancy/lactation, and history of psychosis. The patient was asked not to change her pain medication from the start of the clinical case study until completion of follow up.

### Treatment:

The treatment consisted of a single dose of 100 mg S-ketamine Buccal Mucoadhesive Tablet.

### Measurements:

The primary outcome measure of the study was pain relief as measured by the 10-point NRS ranging from 0 (no pain) to 10 (worst pain), measured 10 times (t=0, 20, 30, 45, 60, 75, 90, 120, 150, 180 minutes) prior start of the treatment and following the administration of the S-ketamine

### Mucoadhesive Tablet.

Secondary outcome parameters were psychotropic side effects, nausea, and headache, all scored on a range from 0 (not present) to 10 (maximal presence).

### Results:

S-Ketamine produced a reduction in pain score from 8 to 2 on treatment day one.

Prior to S-ketamine administration, a base pain score was obtained using a Visual Analogue

Scale (VAS). This was recorded at t=20, 30, 45, 60, 75, 90, 120, 150 and 180 minutes following the administration of S-ketamine.

### VAS-Measurements:

| Time (minutes) | NRS Pain Score |
|---|---|
| 0 | 8 prior to administration |
| 20 | 8 |
| 30 | 5 |
| 45 | 4 |
| 60 | 3 |
| 75 | 2 |
| 90 | 2 |
| 120 | 2 |
| 150 | 2 |
| 180 | 2 |

No side effects have been observed

It is surprising and unexpected that the reduction of the Spontaneous Fibromyalgia Pain occurred at the time course according to the pharmacokinetic profile of S-ketamine and S-norketamine.

It is surprising and unexpected that the side effect profile was minimized because the active ingredient S-ketamine was not metabolized immediately in the liver upon the transmucosal absorption. This will have positive effects on the side effect profile following the repeated administration of high doses of S-ketamine.

### Example 4

### Break-Through Pain (BTCP)—Treatment with S-Ketamine Buccal Mucoadhesive Tablet (100 mg)

One patient (male, age 63 years) eligible for this clinical case was diagnosed with adeno-coloncarcinoma, previously received chemotherapy with no pathological lab values. The patient was suffering from BTCP with severe intensity. Onset of pain was rapid, reached maximum pain scores within 3 minutes or less of start of BTCP. BTCP attacks showed an average of 20-30 minutes. The frequency averages 6 episodes per day. The patient had pain scores of 8+1-2 (average of 3 days) according to the numerical rating scale (NRS) from 0 to 10, where 0=no pain and 10=worst pain). Patient was asked to give informed consent and not to change his pain medication from the start of the clinical case study until completion of follow up.

### Treatment:

The treatment consisted of a daily single dose of 100 mg S-ketamine Buccal Mucoadhesive Tablet for 3 consecutive days=−0 h-24 h, 24-48H, and 48-72 h)

### Measurements:

The primary outcome measures of the study were:

1. Frequency of the pain episodes per 24 hours period
2. Duration of the BTCP per episode
3. The intensity of BTCP

Pain intensity was measured using the 10-point NRS at t=0 (start of episode), 10, 20, 40, and 60 minutes after start of the BTCP attack.

### Results:

### 1. Frequency of the Pain Episodes:

Day 1: (starting with administration at the start of the 1^{st} BTCP attack): 3 (three) episodes

Day 2: 1 (one) BTCP episode

Day 3: 1 (one) BTCP episode

### 2. Duration of BTCP Attack Per Episode

Day 1: 18 minutes

Day 2: 15 minutes

Day 3: 18 minutes

### 3. Pain Intensity of BTCP

| | NRS | NRS | NRS |
|---|---|---|---|
| Measurment Time | Day 1 (0-24 | Day 2 (24-48 | Day 3 (48-72 |
| [Minutes] | hours) | hours) | hours) |
| 0 (start of BTCP | 8 | 3 | 3 |
| attack) | | | |
| 10 | 6 | 0 | 0 |
| 20 | 4 | 0 | 0 |
| 40 | 4 | 0 | 0 |
| 60 | 2 | 0 | 0 |

It is surprising and unexpected that the course of analgesia correlated with plasma concentrations of S-ketamine. This means that the reduction and/or elimination of the breakthrough pain is highly correlated with the respective combined plasma concentrations of S-ketamine and S-norketamine.

It is surprising and unexpected that the frequency of the pain episodes has been reduced drastically during the course of analgesia and is highly correlated with the respective plasma concentrations of the active agents S-ketamine and S-norketamine following the administration of 100 mg S-ketamine Buccal Mucoadhesive Tablet for 3 consecutive days=−24 h, 24-48H, and 48-72 h)

It is surprising and unexpected that the intensity of breakthrough pain has been reduced drastically during the course of analgesia and is highly correlated with the respective plasma concentrations of the active agents S-ketamine and S-norketamine following the administration of 100 mg S-ketamine Buccal Mucoadhesive Tablet for 3 consecutive days=−24 h, 24-48H, and 48-72 h)

It is surprising and unexpected that the duration of the breakthrough pain per episode has been reduced drastically during the course of analgesia and is highly correlated with the respective plasma concentrations of the active agents S-ketamine and S-norketamine following the administration of 100 mg S-ketamine Buccal Mucoadhesive Tablet for 3 consecutive days=−24 h, 24-48H, and 48-72 h)

It is surprising and unexpected that the side effect profile was minimized because the active ingredient S-ketamine was not metabolized immediately in the liver upon the transmucosal absorption. This will have positive effect on the side effect profile following the repeated administration of high doses of S-ketamine.

### Example 5

### Diabetic Neuropathy—Treatment with S-Ketamine Thin Layer Film (100 Mg)

One patient (male, age of 54 years) eligible for this clinical case was diagnosed with a history of untreated DMT2 (diabetes mellitus type 2). Secondary peripheral distal neuropathy of both feet and legs was among the patient's clinical complaints. Efforts to control the neuropathic pain with NSAIDs were only marginally effective.

### Treatment:

The treatment consisted of a daily single dose of 100 mg S-ketamine Thin Layer Film for 3 consecutive days.

### Measurements:

Pain relief as measured by the NRS Numeric Rating Scale) from 0=no pain to 10=worst pain, measured at t=0 (just prior start to the treatment) and at 30, 60, 120, 180, and 360 minutes following the administration of the S-ketamine Thin Layer Film.

Secondary outcome parameters were psychotropic side effects, nausea, and headache, all scored on a range from 0 (not present) to 10 (maximal presence).

### Results:

The patient reported prompt and profound alleviation of all neuropathic pain.

It is surprising and unexpected that the course of analgesia correlated with plasma concentrations of S-ketamine following the administration of 100 mg S-ketamine Thin Layer Film. This means that the reduction and/or elimination of the Neuropathy pain is highly correlated with the respective combined plasma concentrations of S-ketamine and S-norketamine

It is surprising and unexpected that the side effect profile was minimized because the active ingredient S-ketamine was not metabolized immediately in the liver upon the transmucosal absorption. This will have positive effects on the side effect profile following the repeated administration of high doses of S-ketamine.

### Example 6

### Diabetic Neuropathy Using S-Ketamine Orodispersible Granules (Micropellets)

One patient (male, age 62 years) eligible for this clinical case was diagnosed with a history of untreated diabetes. Secondary peripheral distal neuropathy of both feet and legs was among the patients' clinical complaints. Efforts to control the neuropathic pain by resort to treatment with NSAIDs were only marginally effective.

### Treatment:

The treatment consisted of a daily single dose of 100 mg S-ketamine Orodispersible granules (micropellets) for 3 consecutive days.

### Measurements:

Pain relief as measured by the NRS (numeric Rating Scale) from 0 (no pain) to 10 (worst pain), measured at (t=0 (just prior start of the treatment), 30, 60, 120, 180, and 360 minutes) following the administration of the S-ketamine 100 mg S-ketamine Orodisporsible granules (micropellets).

Secondary outcome parameters were psychotropic side effects, nausea, and headache, all scored on a range from 0 (not present) to 10 (maximal presence).

### Results:

The patient reported prompt and profound alleviation of all neuropathic pain.

It is surprising and unexpected that the course of analgesia correlated with plasma concentrations of S-ketamine following the administration of 100 mg S-ketamine Orodisporsible granules (micropellets). This means that the reduction and/or elimination of the Neuropathy pain is highly correlated with the respective combined plasma concentrations of S-ketamine and S-norketamine

It is surprisingly and unexpected that the side effect profile was minimized because the active ingredient S-ketamine was not metabolized immediately in the liver upon the transmucosal absorption. This will have positive effects on the side effect profile following the repeated administration of high doses of S-ketamine.

### Example 7

### Fast Oral Transmucosal (FOT) Formulation for S-Ketamine

The FOT formulation consists of a combined Mucoadhesive system (mucoadhesive patch/tablet) and orodispersible matrix with S-ketamine (and may enable bidirectional release of the active agent S-ketamine to the buccal mucosa and the cavity mucosa)).

The FOT offer distinct advantages over per oral administration of systemic drug delivery such as possible bypass of the first pass effect and avoidance of presystemic elimination within the GI tract. In addition the absorption of the drug will take place through the lining of the oral cavity which is referred to as the oral mucosa, and includes the buccal, sublingual, gingival, palatal and labial mucosa.

For the prophylaxis and/or reduction and/or elimination of acute and chronic Break-Through Pain, CRPS pain syndromes, posttraumatic syndrome, neuropathic pain the active agent S-ketamine will be absorbed very fast bypassing the first pass effect and resulting on a faster onset of action.

The FOT formulation will allow the highest possible bioavailabilty of S-ketamine.

The FOT system may also deliver in one direction the drug towards the mucosa only with an impermeable product surface exposed to the oral cavity (unidirectional drug delivery) which prevents the drug release in this Mucoadhesive part into oral cavity.

The dissolution or disintegration of the orodispersible matrix with S-ketamine, and with or without the water permeable coating will take place in the oral cavity. Increased sucking and saliva production will cause a desired and uncontrolled swallowing and loss of S-ketamine down the GI tract and the increase of the bioavailabity of the active metabolite S-norketamine.

The combined pharmacokinetic profiles of S-ketamine and the active metabolite S-norketamine could be adjusted to achieve the combined fast and sustained onset of action of their analgesic effects.

Unexpected and surprisingly the Fast Oral Transmucosal Formulations (FOT) of S-ketamine have shown synergistic effects on the increase of the bioavailability and efficacy of S-ketamine and S-norketamine with relatively low inter and intra-variability of the S-ketamine release. In addition the FOT formulation represents an ease of access and avoidance of the hepatic metabolism and as alternative to overcome the limitations of conventional oral drug delivery and parental administration.

FIG. 9 provides a schematic representation of the FOT matrix designed for the combined Mucoadhesive system (mucoadhesive patch/tablet) and orodispersible matrix with S-ketamine (bidirectional release of the active agent S-ketamine to the buccal mucosa and the cavity mucosa). The Bioadhesive matrix and orodispersible have been prepared preferably according to the description of the invention. The two layers are attached to the Inert excipient.

### Example 8

### S-Ketamine Sustained Release Tablets

### Comparative Example 1

This example illustrates the preparation of comparison tablets. The following three components are mixed together and formed into tablets. The amount of each component in each tablet is shown in the following table.

| | |
|---|---|
| S-ketamine | 100.0 mg |
| Heweten 12 (Microcrystalline cellulose) | 100.0 mg |
| Magnesium Stearate | 5.0 mg |

Release of the active S-ketamine from the tablets is then determined, (mean value of n=5) and the release data is shown below:

| T (hours) | 1 | 2 | 3 |
|---|---|---|---|
| At % | 77.5 | 97.5 | 100.0 |
| SD | 5.9 | 1.2 | 1.3 |

At %=the weight percent of active agent released from the pharmaceutical form after T hours, based on the weight of incorporated doses of active agent S-ketamine.
SD=standard deviation

The tablets of comparative example 1 (Example 8) behave the way one would expect them to. On account of the degradation promoting properties of the microcrystalline cellulose, the active agent S-ketamine is rapidly and completely released from the pharmaceutical preparation

### Comparative Example 2

The procedure of Comparative Example 1 is repeated, except that the amount of microcrystalline cellulose in each tablet is decreased, as shown by the following composition of each tablet.

| | | |
|---|---|---|
| S-ketamine | 100.0 | mg |
| Heweten 12 (Microcrystalline cellulose) | 20.0 | mg |
| Magnesium Stearate | 5.0 | mg |

Release of the active S-ketamine from the tablets is then determined, (mean value of n=5) and the release data is shown below:

| T (hours) | 1 | 2 | 3 | 4 | 5 |
|---|---|---|---|---|---|
| At % | 29.5 | 54.5 | 76.0 | 94.2 | 100 |
| SD | 3.9 | 6.2 | 3.3 | 5.4 | 1.2 |

As can be seen by comparing the results of Comparative Examples 1 and 2, as the amount of microcrystalline cellulose (Heweten) in the prescription is decreased, release will be decreased because the minimal solubility of S-ketamine, in this case, is the predominant component in the release pattern of the active agent S-ketamine.

The addition of soluble, swelling controller substances such as polyethylene glycols (PEG), which ordinarily are used as solubilizers or to improve the solubility of low solubility active agents, brings about an unexpected delay in the active agent release of low solubility active agents from the systems mentioned as examples in 1 and 2 as shown by the following examples.

### Comparative Example 3

The procedure of comparative Example 1 is repeated, except that each tablet is made in accordance with the present invention in that each tablet additionally contains polyethylene glycol (PEG 35000) as a swelling controller as shown by the following composition of each tablet.

| | |
|---|---|
| S-ketamine | 100.0 mg |
| Heweten 12 (Microcrystalline cellulose) | 100.0 mg |
| Magnesium Stearate | 5.0 mg |
| PEG 35000 (avg. mol. mass 35000) | 5.0 mg |

The release of the active S-ketamine is determined as in Comparative Example 1. Release of the active S-ketamine from the tablets is then determined, (mean value of n=5) and the release data is shown below:

| | T (hours) | | | | | | | |
|---|---|---|---|---|---|---|---|---|
| | 1 | 2 | 3 | 4 | 5 | 6 | 7 | 8 |
| At % | 33 | 55 | 71 | 84 | 94 | 98 | 100 | 100 |
| SD | 2.8 | 3.4 | 3.9 | 5.3 | 6.6 | 4.5 | 3.2 | 2.9 |

### Comparative Example 4

The procedure of comparative example 1 is repeated, except that each tablet has the following composition:

| | | |
|---|---|---|
| S-ketamine | 100.0 | mg |
| Heweten 12 (Microcrystalline cellulose) | 100.0 | mg |
| Magnesium Stearate | 50.0 | mg |
| PEG 35000 (avg. mol. mass 35000) | 5.0 | mg |

The release of the active S-ketamine is determined as in Comparative Example 1. Release of the active S-ketamine from the tablets is then determined, (mean value of n=5) and the release data is shown below:

| | T (hours) | | | | | | | |
|---|---|---|---|---|---|---|---|---|
| | 1 | 2 | 3 | 4 | 5 | 6 | 7 | 8 |
| At % | 13.5 | 17.6 | 22.5 | 26.3 | 28.6 | 34.2 | 36.4 | 36.8 |
| SD | 2.4 | 4.4 | 3.9 | 6.3 | 4.6 | 4.5 | 3.4 | 4.8 |

As can be seen from the above, when combined with a stronger hydrating adjuvant, even small amounts of microcrystalline cellulose capable of limited swelling can cause a delaying effect for low solubility active agents.

It was surprising that the process according to the invention would yield forms which display very slow and controlled release and, contrary to what was to be expected, do not degrade very rapidly and do not release the active agent for immediate resorption.

### Example 9

### Orodispersible Tablets of S-Ketamine

Formulation of Orodispersible tablets:

Tablet each containing 100 mg S-ketamine Hydrochloride was prepared as per composition in the table below. S-ketamine and excipients including taste masking agent were passed through sieve (#80) to ensure the better mixing. Microcristalline Cellulose was used as a direct compressible vehicle. Super disintegrant such as Sodium Starch Glycolate, Crospovidone and Croscarmellose Sodium were used in different ratios. The powder was compressed with a compression machine equipped with 8 mm round punch by direct compression technique. A minimum of 50 tablets was prepared for each batch.

### Example for Composition of Orodispersible Tablets:

| Ingredients (mg) | F1 | F2 | F3 |
|---|---|---|---|
| S-ketamine | 100 | 100 | 100 |
| Lactose | 100 | 120 | 140 |
| Sodium starch glycolate | 8 | 12 | 16 |
| Microcrystalline cellulose | 100 | 120 | 140 |
| Aspartame | 10 | 10 | 10 |
| Magnesium stearate | 2 | 2 | 2 |
| Talc | 10 | 10 | 10 |
| Total weight | 330 | 374 | 418 |

The prepared compositions disintegrate in less than three minutes and were administered without the simultaneous drinking of a glass of water and without the problem of swallowing.

Other techniques for preparing S-ketamine orodispersible tablets not limited to Freeze drying, moulding, sublimation, spray drying, mass extrusion, direct compression.

### Example 10

### Orodispersible Film of S-Ketamine

Different homogenous S-ketamine orodispersible films (ODF) were prepared; the films are translucent, colourless, thin and soft, and with no spot found on the films. The prepared films were evaluated in terms of competitive bioavailability study and clinical cases in patients. In vitro disintegration time was within 60 seconds to 3 minutes. Presence of moisture in films helps them from becoming dry and brittle due to plasticising effect of water.

It was noticed that the films got hydrated rapidly; and began to dissolute the drug within minutes. This may be due to the water solubility of the drug and the polymer.

The fast-dissolving orodispersible films of S-ketamine prepared using different film-forming materials showed satisfactory drug dissolution, acceptable physico-mechanical characteristics and bioavailability of S-ketamine.

Fast-dissolving films with S-ketamine according to this patent are constituted of plasticized hydrocolloids or blends made thereof. Formulation of these were prepared by the known solvent-casting where the polymer and S-ketamine are dissolved (or dispersed) in a solvent (ethanol or water) and a film is cast by solvent evaporation or by hot melt extrusion. Polyvinyl alcohol, polyvinyl pyrrolidone, maltodextrin, microcrystalline cellulose, Hydroxypropyl methyl cellulose, modified starch, chitosan, gums, or blends of these polymers have been used for the film production.

| Ingredients | F1 | F2 |
|---|---|---|
| Lycoat NG73 (granular hydroxypropyl starch) | 15 | — |
| Hydroxypropyl methyl cellulose | — | 4 |
| Alcohol | 15 | — |
| Propylene glycol | 7.5 | 7.5 |
| Maltodextrin | — | 1.25 |
| Menthol^{b} | 0.5 | 0.5 |
| Distilled Water to | 100 | 100 |

The concentration of the drug was 100 mg/4 cm² of the film.
^{a}The amounts are in grams.
^{b}Added as 1 ml solution in ethyl alcohol.

### Example 11

### Orodispersible Granules (Micro-Pellets) of S-Ketamine

Pellets and micropellets of S-ketamine have been prepared using known process technologies. According to these technologies various S-ketamine layering liquids and coating liquids like solutions, suspensions, emulsions, micro-emulsions as well as hot melts have been applied.

The desired S-ketamine dissolution profiles using the different orodispersible granules (micro-pellets) of S-ketamine have been investigated for:
Immediate release as orodispersible formulation for transmucosal administration
Controlled release S-ketamine formulation in capsules
Pulsatile S-ketamine formulation
Gastro-resistent release of S-ketamine

It is surprisingly and unexpected that the prepared Micropellets of S-ketamine have shown increased surface area as compared to traditional compressed tablets and capsules. This has considerably reduced the time required for disintegration/absorption and has the potential for use in rapidly dispersible tablets.

Furthermore it is surprising and unexpected that the prepared S-ketamine micropellets delivered almost perfectly spherical particles with a very narrow particle size distribution and excellent flow properties.

It is surprising and unexpected that the micro-pellets of S-ketamine have shown excellent flow properties and convenient use for filling capsules and the preparation of parental solutions for intravenous, intramuscular and/or subcutaneous applications.

It is surprising and unexpected that the micro-pellets of S-ketamine have shown excellent physicochemical properties for the preparation of oral dry powder for the use for transmucosal, buccal and/or inhalation and/or intranasal applications.

It is surprising and unexpected that all formulations with S-ketamine according to this invention have not shown any racemisation of the enantiomer S-ketamine.

While the invention has been described and illustrated with reference to certain preferred embodiments thereof, those skilled in the art will appreciate that obvious modifications can be made herein without departing from the spirit and scope of the invention. For example, dosage strength per single unit, effective dosages, the different oral transmucosal formulations, and the specific pharmacological responses may vary depending upon the absorption and pharmacokinetic profiles of S-ketamine and/or S-norketamine and/or other metabolites in blood, CNS and tissues, concomitant medications, as well as the ratios of the agent to particular NMDA and/or other receptors. Such variations contemplated to be within the scope of this application.

Additionally, further experimentation shows that the preferred embodiments of the invention provide surprising and unexpected effects, thereby solving the problem of the invention in a non-obvious fashion.

### Example 12

### Linearity Test for Orodispersible Tablets (ODT), Orodispersible Films (ODF), Thin Layer Films, and Fast Oral Transmucosal (FOT) Compositions of S-Ketamine

Linearity investigations have been performed to determine the linear reportable range of S-ketamine following the oral administration of the different transmucosal S-ketamine formulations according to this invention.

It is surprising and unexpected that the dose linearity was observed for the area under the curve (AUC) and maximal blood concentration (Cmax) for all investigated oral transmucosal formulations.

The dose Linearity of S-ketamine was in the range of 10-300 mg S-ketamine.

1.-52. (canceled)

53. A method for treating a subject for pain comprising oral transmucosal administration of a pharmaceutical composition comprising S-Ketamine, salts and/or derivatives thereof to a subject in need thereof in an amount effective to treat pain.

54. The method of claim 53, wherein the oral transmucosal administration is a transbuccal administration.

55. The method of claim 53, wherein the oral transmucosal administration is a sublingual administration.

56. The method of claim 53, wherein a dry powder is administered orally.

57. The method of claim 53, wherein the composition is administered as a fast oral transmucosal (FOT) composition.

58. The method of claim 57, wherein the transmucosal (FOT) composition is adminstered via a mucoadhesive patch.

59. The method of claim 57, wherein the transmucosal (FOT) composition comprises 3 or more layers comprising an orodispersible matrix with S-ketamine, salts and/or derivatives thereof, an inert central layer and a mucoadhesive layer.

60. The method of claim 59, wherein the transmucosal (FOT) composition comprises S-Ketamine in both the orodispersible matrix and mucoadhesive layer, wherein the S-ketamine, salts and/or derivatives thereof are released bidirectionally.

61. The method of claim 60, wherein the S-ketamine, salts and/or derivatives thereof are released bidirectionally to the buccal mucosa and to the cavity mucosa.

62. The method of claim 53, wherein the composition is administered as a sustained release (SR) composition, and wherein said SR composition comprises S-ketamine, salts and/or derivatives thereof as active agent, one or more swelling agents, one or more lubricants and optionally one or more swelling controllers.

63. The method of claim 62, wherein the SR formulation comprises the following components in the following relative ratios (with respect to mass): active agent 50-150: swelling agent 10-200: lubricant 1-100: swelling controller 0-10.

64. The method of claim 53, wherein the composition is administered as an orodispersible tablet (ODT), wherein said ODT formulation comprises S-ketamine, salts and/or derivatives thereof as active agent, one or more excipients, one or more disintegrants and/or swelling agent, optionally one or more sweeteners, one or more lubricants and optionally one or more fillers.

65. The method of claim 64, wherein the ODT composition comprises the following components in the following relative ratios (with respect to mass): active agent 50-150: excipient 50-200: disintegrant and/or swelling agent 10-200: sweetener 0-20: lubricant 0-10: filler 0-50.

66. The method of claim 53, wherein the composition is administered as an orodispersible films (ODF).

67. The method of claim 66, wherein the ODF formulation comprises S-ketamine, salts and/or derivatives thereof as active agent, one or more modified starches suitable for film coating, one or more alcohols, one or more pharmaceutically accepted solvents, one or more binders, one or more flavouring agents, and preferably water.

68. The method of claim 66, wherein the ODF formulation comprises S-ketamine, salts and/or derivatives thereof as active agent at 10 to 500 mg/4 cm² of the film.

69. The method of claim 68, wherein the ODF formulation comprises S-ketamine, salts and/or derivatives thereof as active agent at 50 to 150 mg/4 cm² of the film.

70. The method of claim 66, wherein the ODF formulation comprises S-ketamine, salts and/or derivatives thereof as, wherein the ODF formulation comprises the following components in the following relative percentages (with respect to mass; active agent is not included in these amounts but is added to the film as described herein): modified starch 2-30: alcohol 0-20: solvent 5-20: binder 0-5: flavouring agent 0-5: water to make up the remaining to 100.

71. The method of claim 53, wherein the composition is administered as ordodispersible granules (micro-pellets).

72. The method of claim 53, wherein the S-ketamine derivative is nor-S-ketamine.

73. The method of claim 53, wherein the S-ketamine derivative is S-Dehydronorketamine.

74. The method of claim 53, wherein the S-ketamine derivative is or (S,S)-6-Hydroxynorketamine.

75. The method of claim 53, wherein the S-ketamine salt is S-Ketamine hydrochloride.

76. The method of claim 53, wherein the S-ketamine salt is a salt of an organic acid, wherein the S-ketamine salt of an organic acid is of an acetic, trifluoroacetic, propionic, succinic, glycolic, stearic, lactic, malic, tartaric, citric, ascorbic, or amino acid salt, wherein the amino acid salt is arginate, asparginate, or glutamate.

77. The method of claim 53, wherein the pain is chronic pain.

78. The method of claim 77, wherein the chronic pain is chronic break-through pain (BTCP).

79. The method of claim 53, wherein the pain is complex regional pain syndrome (CRPS).

80. The method of claim 53, wherein the pain is refractory cancer pain.

81. The method of claim 53, wherein the pain is neuropathic pain.

82. The method of claim 53, wherein the pain is post traumatic syndrome pain (PTSD).

83. The method of claim 53, wherein the pain is ischaematic limb pain.

84. The method of claim 53, wherein the pain is acute pain.

85. The method of claim 57, wherein the OFT composition is administered at a single dose of between 10 to 200 mg of S-Ketamine.

86. The method of claim 85, wherein the OFT composition is administered at a single dose of between 40 to 120 mg of S-Ketamine.

87. The method of claim 62, wherein the SR composition is administered at a single dose of between 100 to 500 mg of S-Ketamine.

88. The method of claim 62, wherein the SR composition is administered at a single dose providing between 10 to 50 mg of S-Ketamine per hour for 8 to 16 hours.

89. The method of claim 53, wherein the composition is administered in combination with opioid therapy in cancer patients with pain.

90. The method of claim 53, wherein an effective amount of a second agent is administered, wherein said agent is selected from the group consisting of a pharmaceutical NMDA receptor antagonist, analgesic drug, narcotic analgesic opioid, a non-steroidal anti-inflammatory analgesic (NSAIA), antidepressant, neuroleptic agent, anticonvulsant, a mood stabilizer, an antipsychotic agent, anticancer agent and benzodiazepine.
